(19) United States
(12) Patent Application Publication
(10) Pub. No.: US 2009/0004675 A1
(43) Pub. Date: Jan. 1, 2009

(54) Method of Identifying Compounds for Bacterial Growth Modulation

(75) Inventors: Michael E. O'Donnell, Hastings-On-Hudson, NY (US); Roxana Georgescu, Yonkers, NY (US)

Correspondence Address: NIXON PEABODY LLP - PATENT GROUP, 1100 CLINTON SQUARE, ROCHESTER, NY, 14604, US

(73) Assignee: THE ROCKEFELLER UNIVERSITY, New York, NY (US)

(21) Appl. No.: 11/718,581

(22) PCT Filed: Nov. 3, 2005
(86) PCT No.: PCT/US05/39997

§ 371 Date: Apr. 7, 2008

Related U.S. Application Data
(60) Provisional application No. 60/624,932, filed on Nov. 4, 2004.

(51) Int. Cl. G01N 33/573; G01N 33/53

(52) U.S. Cl. 435/7.4; 435/7.1

(57) ABSTRACT

The present invention relates to a method of identifying a candidate compound for modulating bacterial growth. This the method involves providing a β clamp peptide from a bacterial replicase, providing a second peptide that binds to at least one amino acid of SEQ ID NO:9 that is not designated X, wherein the second peptide does not exhibit polymerase activity, and providing a test compound. The β clamp peptide and the second peptide are contacted with the test compound, and the level of binding between the β clamp peptide and the second peptide in the presence of the test compound is determined. The level of binding between the β clamp peptide and the second peptide in the presence of the test compound is then compared to a control that does not contain the test compound. A test compound that alters the level of binding between the β clamp peptide and the second peptide compared to the control is a candidate compound for modulating bacterial growth.

This application claims the benefit of U.S. Provisional Patent Application Ser. No. 60/624,932, filed Nov. 4, 2004, which is hereby incorporated in its entirety.

The present invention was made with funding from National Institutes of Health Grant No. GM38839. The United Stated Government may have certain rights in this invention.

### FIELD OF THE INVENTION

The present invention relates to a method of identifying compounds for bacterial growth modulation.

### BACKGROUND OF THE INVENTION

All forms of life must duplicate the genetic material to propagate the species. The process by which the DNA in a chromosome is duplicated is called replication. The replication process is performed by numerous proteins that coordinate their actions to smoothly duplicate the DNA. The main protein actors are as follows (reviewed in Kornberg, et al., DNA Replication, Second Edition, New York: W.H. Freeman and Company, pp. 165-194 (1992)). A helicase uses the energy of ATP hydrolysis to unwind the two DNA strands of the double helix. Two copies of the DNA polymerase use each “daughter” strand as a template to convert them into two new duplexes. The DNA polymerase acts by polymerizing the four monomer unit building blocks of DNA (the 4 dNTPs, or deoxynucleoside triphosphates are: dATP, dCTP, dGTP, dTTP). The polymerase rides along one strand of DNA using it as a template that dictates the sequence in which the monomer blocks are to be polymerized. Sometimes the DNA polymerase makes a mistake and includes an incorrect nucleotide (e.g., A instead of G). A proofreading exonuclease examines the polymer as it is made and excises building blocks that have been improperly inserted in the polymer.

Duplex DNA is composed of two strands that are oriented antiparallel to one another, one being oriented 3′-5′ and the other 5′ to 3′. As the helicase unwinds the duplex, the DNA polymerase moves continuously forward with the helicase on one strand (called the leading strand). However, due to the fact that DNA polymerases can only extend the DNA forward from a 3′ terminus, the polymerase on the other strand extends DNA in the opposite direction of DNA unwinding (called the lagging strand). This necessitates a discontinuous ratcheting motion on the lagging strand in which the DNA is made as a series of Okazaki fragments. DNA polymerases cannot initiate DNA synthesis de novo, but require a primed site (i.e. a short duplex region). This job is fulfilled by primase, a specialized RNA polymerase, that synthesizes short RNA primers on the lagging strand. The primed sites are extended by DNA polymerase. A single stranded DNA binding protein (SSB) is also needed; it operates on the lagging strand. The function of SSB is to coat single stranded DNA (ssDNA), thereby melting short hairpin duplexes that would otherwise impede DNA synthesis by DNA polymerase.

The replication process is best understood for the Gram negative bacterium, Escherichia coli, (reviewed in Kelman, et al., “DNA Polymerase III Holoenzyme: Structure and Function of Chromosomal Replicating Machine,” Annu. Rev. Biochem., 64:171-200 (1995); Marians, K. J., “Prokaryotic DNA Replication,” Annu. Rev. Biochem., 61:673-719 (1992); McHenry, C. S., “DNA Polymerase III Holoenzyme: Components, Structure, and Mechanism of a True Replicative Complex,” J. Bio. Chem., 266:19127-19130 (1991). The helicase of E. coli is encoded by the dnaB gene and is called the DnaB-helicase. The helicase contacts the DNA polymerase. This contact is necessary for the helicase to achieve the catalytic efficiency needed to replicate a chromosome (Kim, et. al., “Coupling of a Replicative Polymerase and Helicase: A tau-DnaB Interaction Mediates Rapid Replication Fork Movement,” Cell, 84:643-650 (1996)). The primase of E. coli is a small RNA polymerase (product of the dnaG gene) and it makes a short 10-12 nucleotide RNA to prime elongation by the polymerase.

The chromosomal replicating DNA polymerase of E. coli and other prokaryotes is processive; it remains continuously associated with the DNA template as they link monomer units (dNTPs) together. This catalytic efficiency can be manifest in vitro by the ability to extend a single primer around a circular single stranded DNA (ssDNA) of over 5,000 nucleotide units in length. The bacterial chromosomal DNA polymerases will be referred to here as replicases to distinguish them from DNA polymerases that function in other DNA metabolic processes and are far less processive.

The replicases consist of three functional components, a sliding clamp protein, a ATP requiring clamp loader protein complex, and the DNA polymerase. In these systems, the sliding clamp protein is an oligomer in the shape of a ring. The clamp loader is a multiprotein complex which uses ATP to assemble the clamp around DNA. The DNA polymerase then binds the clamp which tethers the polymerase to DNA for high processivity. In this application, any replicase that uses a minimum of three components (i.e. clamp, clamp loader, and DNA polymerase) will be referred to as either a DNA polymerase III or Pol C type replicase.

The E. coli replicase is also called DNA polymerase III holoenzyme. The holoenzyme is a single multiprotein particle that contains all the components and, therefore, is composed of 10 different proteins. This holoenzyme is suborganized into three functional components called: 1) Pol III core (DNA polymerase); 2) tau/gamma complex (clamp loader); and 3) beta subunit (sliding clamp). The DNA polymerase III “core” is a tightly associated complex containing one each of the following three subunits: 1) the alpha subunit which is the actual DNA polymerase (129 kDa); 2) the epsilon subunit (28 kDa) which contains the proofreading 3′-5′ exonuclease activity; and 3) the theta subunit which has an unknown function. The tau/gamma complex is the clamp loader and contains the following subunits: tau, gamma, delta, delta prime, chi, and psi (U.S. Pat. No.5,583,026 to O'Donnell). The beta subunit is a homodimer and forms the ring shaped sliding clamp. These components associate to form the holoenzyme and the entire holoenzyme can be assembled in vitro from 10 isolated pure subunits (U.S. Pat. No. 5,583,026 to O'Donnell; U.S. Pat. No. 5,668,004 to O'Donnell). The tau subunit, encoded by the same gene that encodes gamma (dnaX), acts as a glue to hold two cores together with one gamma complex. This subassembly is called DNA polymerase III star (Pol III*). One beta ring interacts with each core in Pol III* to form DNA polymerase III holoenzyme.

During replication, the two cores in the holoenzyme act coordinately to synthesize both strands of DNA in a duplex chromosome. At the replication fork, DNA polymerase III holoenzyme physically interacts with the DnaB helicase through the tau subunit to form a yet larger protein complex termed the “replisome” (Kim, et. al., “Coupling of a Replicative Polymerase and Helicase: A tau-DnaB Interaction Mediates Rapid Replication Fork Movement,” Cell, 84:643-650 (1996); Yuzhakov, et. al., “Replisome Assembly Reveals the Basis for Asymmetric Function in Leading and Lagging Strand Replication,” Cell, 86:877-886 (1996)). The primase repeatedly contacts the helicase during replication fork movement to synthesize RNA primers on the lagging strand (Marians, K. J., “Prokaryotic DNA Replication,” Annu. Rev. Biochem., 61:673-719 (1992)).

In addition, new genes from Gram positive bacteria (e.g., Staphylococcus aureus and Streptococcus pyogenes) were identified (Bruck I. et al., “The DNA Replication Machine of a Gram-Positive Organism,” J. Biol. Chem., 275:28971-28983, (2000)). The Gram positive class of bacteria includes some of the worst human pathogens, such as Staphylococcus aureus, Streptococcus pneumoniae, Streptococcus pyogenes, Enterococcus faecalis, and Mycobacterium tuberculosis (Youmans, et. al., The Biological and Clinical Basis of Infectious Disease (1985)). It was demonstrated that the Pol C polymerase of Gram positive bacteria binds the β clamp for high processivity. Gram positive cells, like B. subtilis and S. aureus, also have a dnaN gene encoding beta (Alonso, et al., “Nucleotide Sequence of the recF Gene Cluster From Staphylococcus aureus and Complementation Analysis in Bacillus subtilis recF Mutants,” Mol. Gen. Genet., 246:680-686 (1995); Alonso, et al., “Nucleotide Sequence of the recF Gene Cluster From Staphylococcus aureus and Complementation Analysis in Bacillus subtilis recF Mutants,” Mol. Gen. Genet., 248:635-636 (1995)). This Gram positive beta subunit functions in replication with Pol C as a sliding clamp (Klemperer, N., et. al. “Cross Utilization of the Beta Sliding Clamp by Replicative Polymerases of Evolutionary Divergent Organisms,” J. Biol. Chem., 275:26136-26143 (2000); Bruck, I. et al., “Reconstitution of the DNA Replication Apparatus of Streptococcus Pyogenes, a Gram Positive Organism,” J. Biol. Chem., 275:28971-28983 (2000)). Furthermore, genes from Gram positive cells encoding functional homologues of the E. coli clamp loader τ(γ)δδ′ subunits have been identified and characterized functionally (Bruck, I. et al., “Reconstitution of the DNA Replication Apparatus of Streptococcus Pyogenes, a Gram Positive Organism,” J. Biol. Chem., 275:28971-28983 (2000)).

Pol C (about 165 kDa) shares homology with both the E. coli Pol III alpha (about 129 kDa) and ε (about 27 kDa) subunits of E. coli, and, accordingly, Pol C contains both DNA polymerase and proofreading 3′-5′ exonuclease activity (Gass, et al., “Further Genetic and Enzymological Characterization of the Three Bacillus subtilis Deoxyribonucleic Acid Polymerases,” J. Biol. Chem., 248:7688-7700 (1973); Ganesan, et. al.; “DNA Replication in a Polymerase I Deficient Mutant and the Identification of DNA Polymerases II and III in Bacillus subtilis,” Biochem. And Biophy. Res. Commun., 50:155-163 (1973); Ott, et. al.; “Cloning and Characterization of the Pol C Region of Bacillus subtilis,” J. Bacteriol., 165:951-957 (1986); Barnes, et. al., “Localization of the Exonuclease and Polymerase Domains of Bacillus subtilis DNA Polymerase III,” Gene, 111:43-49 (1992); Barnes, et. al., “The 3′-5′ Exonuclease Site of DNA Polymerase III From Gram-positive Bacteria: Definition of a Novel Motif Structure,” Gene” 165:45-50 (1995); and Barnes, et al., “Purification of DNA Polymerase III of Gram-positive Bacteria,” Methods in Enzy., 262:35-42 (1995)). The S. aureus Pol C gene has been sequenced, expressed in E. coli, and purified; it contains polymerase and 3′-5′ exonuclease activity (Pacitti, et. al., “Characterization and Overexpression of the Gene Encoding Staphylococcus aureus DNA Polymerase III,” Gene, 165:51-56 (1995)). Although this Pol C is essential to cell growth (Clements, et. al., “Inhibition of Bacillus subtilis Deoxyribonucleic Acid Polymerase III by Phenylhydrazinopyrimidines: Demonstration of a Drug-induced Deoxyribonucleic Acid-Enzyme Complex,” J. Biol. Chem., 250:522-526 (1975); Cozzarelli, et al., “Mutational Alteraction of Bacillus subtilis DNA Polymerase III to Hydroxyphenylazopyrimidine Resistance: Polymerase III is Necessary for DNA Replication,” Biochem. and Biophy. Res. Commun., 51:151-157 (1973); Low, et. al., “Mechanism of Inhibition of Bacillus subtilis DNA Polymerase III by the Arylhydrazinopyrimidine Antimicrobial Agents,” Proc. Natl. Acad. Sci. USA, 71:2973-2977 (1974)), there is still another DNA polymerase that is essential to the cell. Gram positive cells contain a second essential DNA polymerase with homology to E. coli α, and applicants' previous work has shown that this “DnaE” polymerase also functions with the β clamp (Bruck I. et al., “The DNA Replication Machine of a Gram-Positive Organism,” J Biol. Chem., 275:28971-28983, (2000)).

Antibacterial drugs are important to human health. However, numerous strains of resistant bacteria are developing, and it is widely understood that new drugs which inhibit novel targets are needed. This is particularly true with regard to members of the Staphylococcus genus in view of the emergence of drug resistant strains of these organisms. For example, Staphylococcus aureus has successfully mutated to become resistant to all common antibiotics.

The “target” protein(s) of an antibiotic drug is generally involved in a critical cell function, such that blocking its action with a drug causes the pathogenic cell to die or no longer proliferate. Current antibiotics are directed to very few targets. These include membrane synthesis proteins (e.g., vancomycin, penicillin, and its derivatives such as ampicillin, amoxicillin, and cephalosporin), the ribosome machinery (tetracycline, chloramphenicol, azithromycin, and the aminoglycosides: kanamycin, neomycin, gentamicin, streptomycin), RNA polymerase (rifampimycin), and DNA topoisomerases (novobiocin, quinolones, and fluoroquinolones).

DNA replication of the chromosome is an essential life process that requires a DNA replicase machinery. Hence, the replicase would likely be a good target of antimicrobial drugs. In fact, the polymerase subunit of the DNA replicase has been validated as a target of the HP-ura class of antimicrobials which target Gram positive replicase (Kornberg, et al., DNA Replication, Second Edition, New York: W.H. Freeman and Company, pp. 165-194 (1992); Clements, et. al., “Inhibition of Bacillus subtilis Deoxyribonucleic Acid Polymerase III by Phenylhydrazinopyrimidines: Demonstration of a Drug-induced Deoxyribonucleic Acid-Enzyme Complex,” J. Biol. Chem., 250:522-526 (1975); Cozzarelli, et al., “Mutational Alteraction of Bacillus subtilis DNA Polymerase III to Hydroxyphenylazopyrimidine Resistance: Polymerase III is Necessary for DNA Replication,” Biochem. And Biophy. Res. Commun., 51:151-157 (1973); Low, et. al., “Mechanism of Inhibition of Bacillus subtilis DNA Polymerase III by the Arylhydrazinopyrimidine Antimicrobial Agents,” Proc. Natl. Acad. Sci. USA, 71:2973-2977 (1974). However, the HP-ura chemical class has low solubility in general and have not found application as a applied drug. DNA polymerases are also validated targets in eukaryotic viral systems. For example, AZT inhibits HIV reverse transcriptase, acyclovir inhibits HSV1 DNA polymerase, and cidiovir targets pox DNA polymerase.

The present invention is directed to an assay for meeting the need in the art for new antibacterial agents.

### SUMMARY OF THE INVENTION

The present invention relates to a method of identifying a candidate compound for modulating bacterial growth. This the method involves providing a β clamp peptide from a bacterial replicase, providing a second peptide that binds to at least one amino acid of SEQ ID NO:9 that is not designated X, wherein the second peptide does not exhibit polymerase activity, and providing a test compound. The β clamp peptide and the second peptide are contacted with the test compound, and the level of binding between the β clamp peptide and the second peptide in the presence of the test compound is determined. The level of binding between the β clamp peptide and the second peptide in the presence of the test compound is then compared to a control that does not contain the test compound. A test compound that alters the level of binding between the β clamp peptide and the second peptide compared to the control is a candidate compound for modulating bacterial growth.

The present invention relies on the ability of the last several C-terminal residues of E. coli α to bind to the E. coli β clamp. Furthermore, a C-terminal peptide corresponding to the C-terminal residues of Pol C from Gram positive Streptococcus pyogenes binds to β clamps from Gram negative E. coli and Gram positive Streptococcus pyogenes, and the peptide from Staphylococcus aureus Pol C binds E. coli β and S. pyogenes β.

The polymerase-β connection point is well conserved in bacteria and should form the basis for a broad spectrum antibiotic. The present invention demonstrates that a peptide displacement assay can be used to screen a chemical library for chemical compounds that can displace the polymerase C-terminal peptide from the β clamp. Alternatively, chemical compounds that disrupt this polymerase-clamp interaction and inhibit cell growth can also be identified.

Compounds that bind to the β clamps of Gram positive and Gram negative bacteria can be identified in accordance with the present invention. Subsets of these compounds inhibit DNA replication in assays containing the entire DNA polymerase, the β clamp, and clamp loader and/or prevent bacterial growth in liquid culture.

As described above, bacterial replicases are composed of several proteins, most of which are encoded by essential genes. Notable among these are the circular β sliding clamp which encircles DNA and binds the replicative DNA polymerase, holding it to DNA for high processivity during DNA polymerization. The circular clamp is loaded onto DNA by a multiprotein clamp loader that uses ATP to open and close the ring around the primed site.

An essential attachment point of the E. coli Pol III polymerase to the E. coli clamp occurs between the C-terminal residues of the DNA polymerase and the β clamp (López de Saro, F. J., et. al. Competitive Processivity-Clamp Usage by DNA Polymerase During DNA Replication and Repair. EMBO J., 22:6408-6418 (2003), which is hereby incorporated by reference in its entirety). Hence, a small-molecule that disrupts the polymerase connection to the essential β clamp processivity factor should prevent DNA synthesis and bacterial cell growth.

The β clamp represents a new and novel antibacterial drug target. β clamps are conserved across diverse Gram positive and Gram negative bacteria. In fact, the β sliding clamp processivity factor of the gram negative E. coli organism has been shown to bind and function with gram positive Pol C replicases. This implies a high conservation of this important protein-protein connection. Hence, a chemical compound that binds in this pocket may exhibit a broad spectrum cell growth inhibition. These bacterial clamps display no homology to the eukaryotic PCNA clamp. Hence a chemical that binds β should not bind PCNA and effect a eukaryotic replicase.

A powerful approach to discovery of a new drug is to screen large chemical libraries in functional assays to identify compounds that inhibit the target protein. These candidate pharmaceuticals are then chemically modified to optimize their potency, breadth of antibiotic spectrum, performance in animal models, non toxicity, and, finally, use in clinical trials. The assay of the present invention is a selective and robust assay which reliably screens a large chemical library. This assay is insensitive to most chemical compounds in the concentration range normally used in the drug discovery process and does not show inhibition by antibiotics known to target proteins in processes outside of replication.

The present invention relates to various inhibitors of bacteria. The invention provides an antibacterial compound and efficient methods of identifying pharmacological agents or lead compounds for agents active at preventing the replicase Pol III, or Gram Positive replicase, Pol C, from binding the β clamp. It also provides methods for determining which compounds can shut down DNA replication in vitro. The assay methods are amenable to automated, cost-effective high throughput screening of libraries for lead compounds. The invention also provides methods to obtain the crystal structure of compounds bound to β.

Identified reagents find use in the pharmaceutical industries for animal and human trials; for example, the reagents may be derivatized and rescreened in in vitro and in vivo assays to optimize activity and minimize toxicity for pharmaceutical development. Target therapeutic indications are limited only in that the target cellular function can be subject to modulation, usually inhibition, by disruption of a complex comprising a replication polymerase protein and the β clamp processivity factor. Target indications may include arresting cell growth or causing cell death resulting in recovery from the bacterial infection in animal studies.

### BRIEF DESCRIPTION OF THE DRAWINGS

FIGS. 1A-C demonstrates that polymerase peptides corresponding to the C-terminal 20 residues bind β clamps from various species. In FIG. 1A, the E. coli β clamp binds peptides derived from E. coli Pol III α subunit, S. pyogenes Pol C, and S. aureus Pol C. The assays corresponding to the data in the Table of FIG. 1A, use E. coli β labeled with an Oregon Green fluorophore. Peptides of various polymerases were titrated into the labeled β, and the K_{d} values were obtained from the resulting fluorescent intensity changes. All other plots utilize rhodamine-labeled polymerase peptide from E. coli α (FIG. 1A), S. pyogenes Pol C (FIG. 1B), S. aureus Pol C (FIG. 1C). β clamp from the organisms indicated are titrated into the fluorescent peptide.

FIGS. 2A-C show that peptides derived from the C-termini of the E. coli Pol III, S. pyogenes Pol C, and S. aureus Pol C inhibit replication by all three replicases.

FIG. 3 shows a 384 well plate using the peptide displacement from β assay. The fluorescent anisotropy change of E. coli Pol III rhodamine-labeled peptide is monitored in the presence of various chemicals.

FIG. 4A-B show an IC₅₀ titration of a chemical “hit” into the peptide displacement fluorescent anisotropy assay using rhodamine labeled E. coli Pol III peptide (FIG. 4A) and E. coli (FIG. 4B).

FIG. 5 shows that a subset of compounds inhibit the E. coli Pol III holoenzyme in vitro.

FIG. 6 shows that a subset of compounds that bind E. coli β also bind S. pyogenes β. The fluorescence anisotropy peptide displacement assay is used with S. pyogenes β and a S. pyogenes Pol C rhodamine-labeled peptide to evaluate compounds that scored positive in displacing the rhodamine E. coli Pol III peptide from E. coli β.

FIG. 7 shows the DNA synthesis replication assay in which a compound is titrated into the in vitro holoenzyme replicase systems of E. coli and S. pyogenes using the β clamp, clamp loader, and DNA polymerase. A eukaryotic specificity control using the yeast replicase, Pol δ, RFC clamp loader, and PCNA clamp is also shown. The substrate is M13mp18 ssDNA coated with E. coli SSB and primed with a synthetic DNA primer.

FIG. 8 shows the DNA synthesis replication assay in which a compound is titrated into the E. coli and Streptococcus pyogenes holoenzyme replicase using the β clamp, clamp loader, and DNA polymerase. The yeast Pol δ, RFC, PCNA replicase system is shown as a control. The substrate is M13mp18 ssDNA coated with E. coli SSB and primed with a synthetic DNA primer.

FIGS. 9A-B show E. coli cell growth (FIG. 9A) and Strep. aureus cell growth (FIG. 9B) in the presence of various compounds that scored positive in the β clamp peptide displacement assay.

FIG. 10 shows the crystal structure of E. coli α subunit peptide bound to one half of the β clamp. Peptide also binds the same spot in the other β monomer.

FIG. 11 shows the crystal structure of S. aureus PolC peptide bound to β.

FIG. 12 shows the co-crystal structure of a compound with the β clamp showing the location of a compound bound to one half of the clamp. This structure has been refined to 1.6 angstrom, revealing the amino acid side chains in β that interact with the compound. The compound also binds the same spot in the other in the other β monomer.

FIGS. 13A-E show an alignment of β sequences including those from pathogenic Gram negative and Gram positive organisms. In particular, with regard to the Gram negative bacteria, the amino acid sequences for the β clamps from the following bacteria are shown: Escherichia coli (SEQ ID NO: 1), Pseudomonas aeruginosa (SEQ ID NO: 2), and Salmonella typhimurium (SEQ ID NO: 3). As to the Gram negative bacteria, the amino acid sequences for the following bacteria are shown: Staphylococcus aureus (SEQ ID NO: 4), Streptococcus pneumoniae (SEQ ID NO: 5), Streptococcus pyogenes (SEQ ID NO: 6), Enterocccus faecalis (SEQ ID NO: 7), and Mycobacterium tuberculosis (SEQ ID NO: 8). Positions corresponding to the consensus sequence for polymerase peptide binding are shown at the top of the alignments.

### DETAILED DESCRIPTION OF THE INVENTION

The present invention relates to a method of identifying a candidate compound for modulating bacterial growth. This the method involves providing β clamp peptide from a bacterial replicase, providing a second peptide that binds to at least one amino acid of SEQ ID NO:9 that is not designated X, wherein the second peptide does not exhibit polymerase activity, and providing a test compound. The β clamp peptide and the second peptide are contacted with the test compound, and the level of binding between the β clamp peptide and the second peptide in the presence of the test compound is determined. The level of binding between the β clamp peptide and the second peptide in the presence of the test compound is then compared to a control that does not contain the test compound. A test compound that alters the level of binding between the β clamp peptide and the second peptide compared to the control is a candidate compound for modulating bacterial growth.

In carrying out the method of the present invention, the second peptide can bind to at least one residue of SEQ ID NO:9 that is designated X. Further, the β clamp peptide can be a full length β clamp protein.

When the method of the present invention determines that the level of binding between the β clamp peptide and the second peptide is decreased in the presence of the test compound, the test compound is a candidate compound for modulating bacterial growth. Alternatively, when the level of binding between the β clamp protein and the second peptide is increased in the presence of the test compound, the test compound is a candidate compound for modulating bacterial growth. In particular, the method of the present invention can be carried out such that the comparing step determines whether the test compound inhibits binding between the β clamp peptide and the second peptide or whether the test compound promotes binding between the β clamp peptide and the second peptide.

The compounds which are identified as disrupting binding between the β clamp protein and the peptide which is capable of binding to a portion of the β clamp protein can be administered to a subject to treat or prevent bacterial infection. Such compounds can be administered by conventional modes of administration and can formulated in conventional forms.

The ratio of the second peptide and the β clamp protein should be arranged such that most of the second peptide is bound to the β clamp. Suitable ratios that will suffice, provided the concentration of the β clamp is near the K_{d}, or higher than the K_{d}, are 1:1 (second peptide: β clamp), 0.75:1, 0.5:1, 0.25:1, 0.1:1, and 0.05:1.

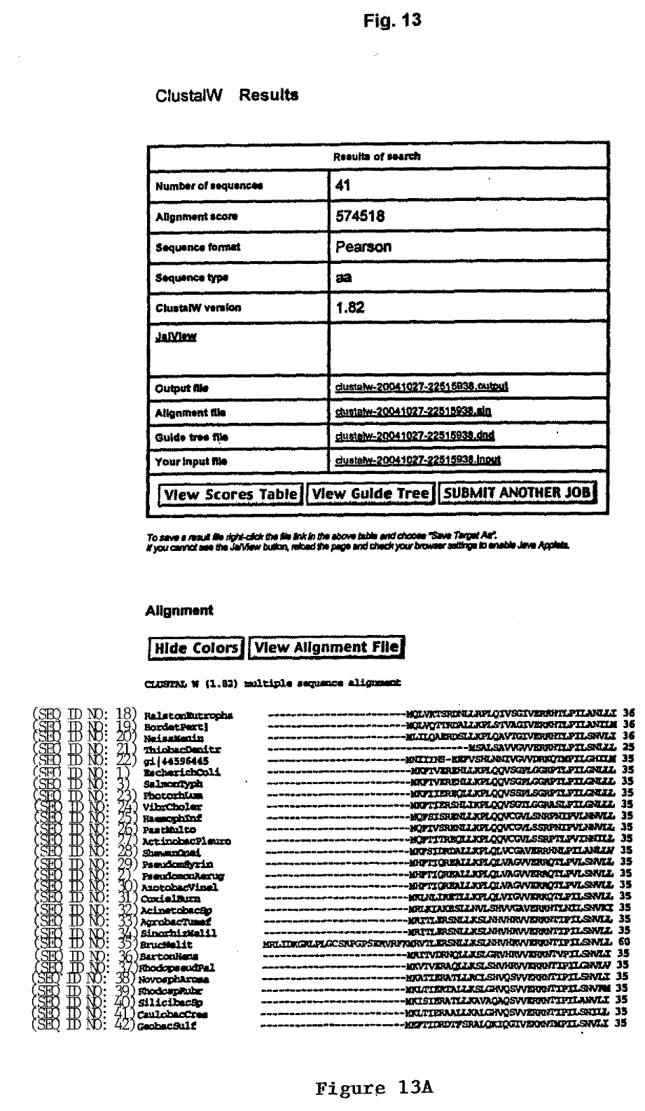

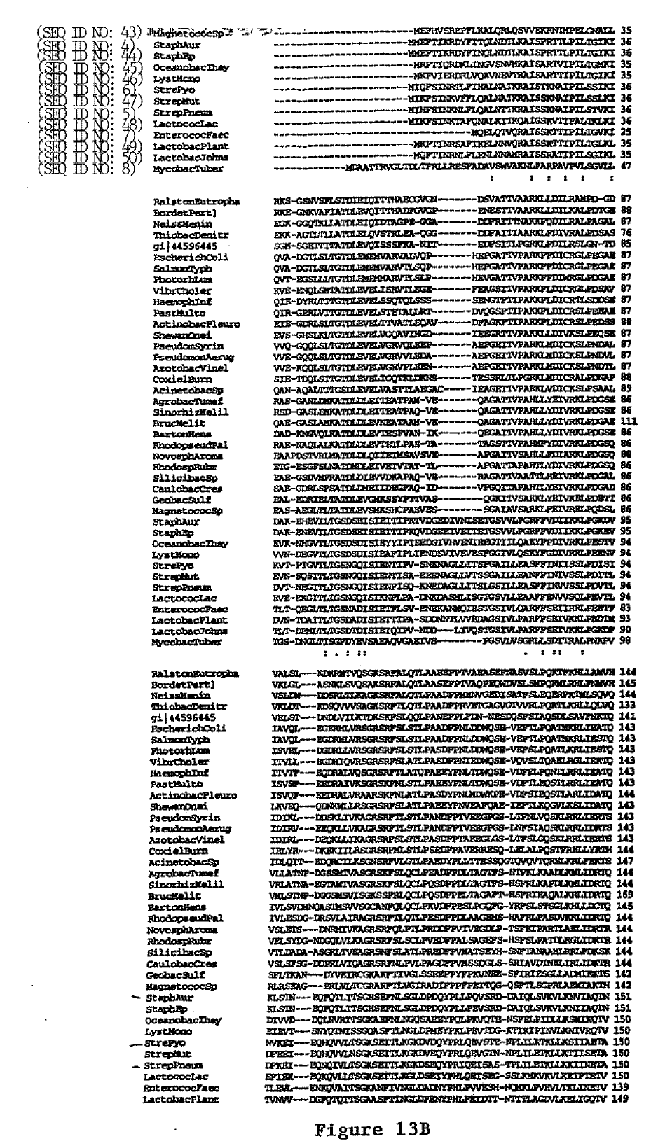

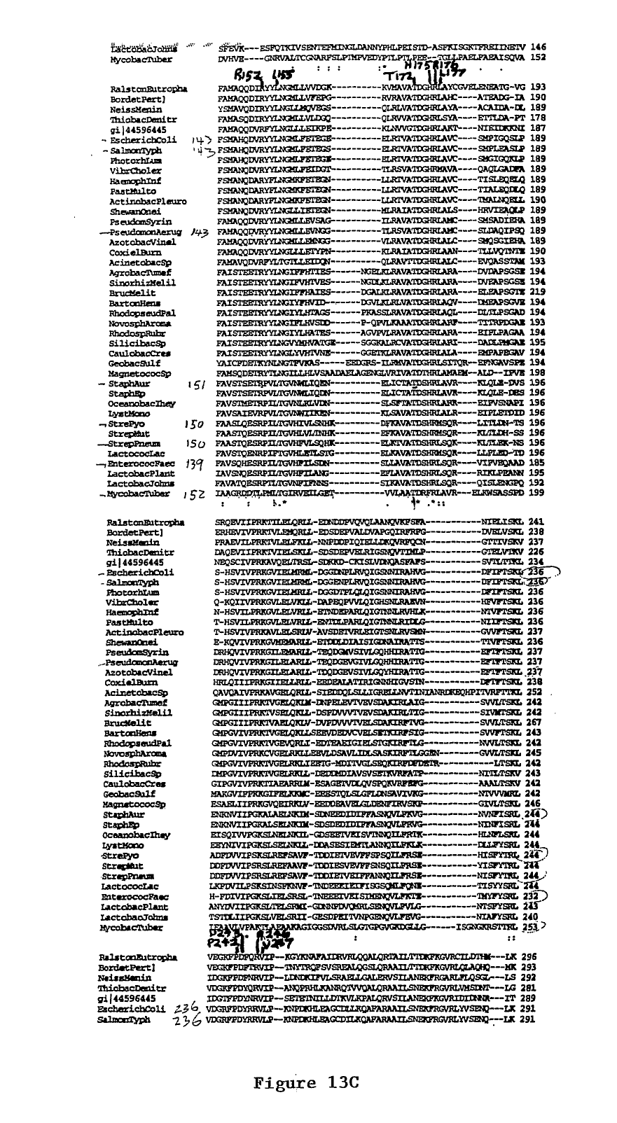

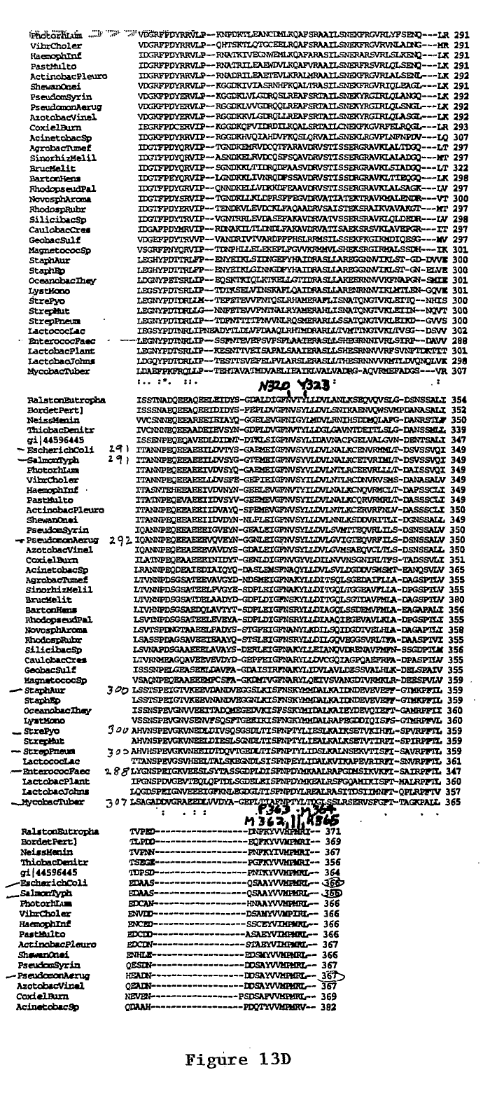

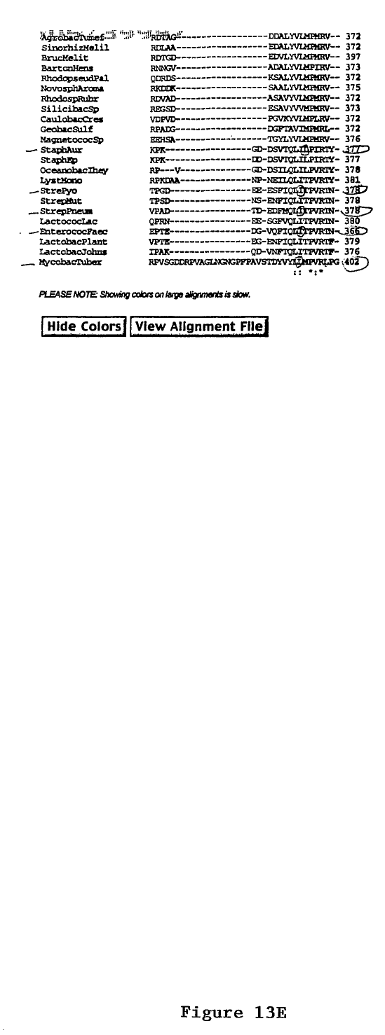

The amino acid sequence of various β clamp proteins are well known, as set forth in FIG. 13.

For example, the β clamp protein from various Gram negative bacteria can be used in practicing the method of the present invention. For example, the β clamp from Escherichia coli (SEQ ID NO: 1), Pseudomonas aeruginosa (SEQ ID NO: 2), or Salmonella typhimurium (SEQ ID NO: 3) can be used in accordance with the present invention.

Alternatively, the β clamp protein from various Gram positive bacteria can be used in practicing the method of the present invention. For example, the β clamp from Staphylococcus aureus (SEQ ID NO: 4), Streptococcus pneumoniae (SEQ ID NO: 5), Streptococcus pyogenes (SEQ ID NO: 6), Enterococcus faecalis (SEQ ID NO: 7), and Mycobacterium tuberculosis (SEQ ID NO: 8) can be used in accordance with the present invention.

Alignment of the β clamp proteins in the two preceding paragraphs reveals a conserved β clamp consensus protein (SEQ ID NO: 9) as follows (see also FIG. 13):

R-X₂-L-X₁₆₋₂₆-T-X₂-H-R-L-X₆₄₋₇₆-P-D/K-X₂-R-X₀₋₁-V/L-X₇₂₋₇₅-N-X₂-Y-X₃₈₋₅₅-M/L/I-X₀₋₁-P-M/I/V-X₂-R

where X is any amino acid. Table 1 (below) shows how the portions of the β clamp protein amino acid sequences for the various Gram positive and Gram negative bacteria used to establish the conserved β clamp consensus protein correspond to SEQ ID NO: 9.

TABLE 1

| Consensus | E. | Sal. | P. | Staph. | Strep. | Strep. | Entero. | Myco. |
|---|---|---|---|---|---|---|---|---|
| (SEQ ID NO: 9) | coli | typ | aer. | aur. | pyo. | pneu. | F. | tuber. |
| R | R152 | R152 | R152 | R160 | R159 | R159 | R148 | L161 |
| X₂ | X₂ | X₂ | X₂ | X₂ | X₂ | X₂ | X₂ | X₂ |
| L | L155 | L155 | L155 | L163 | L162 | L162 | L151 | L164 |
| X₁₆₋₂₆ | X₁₆ | X₁₆ | X₁₆ | X₁₆ | X₁₇ | X₁₇ | X₁₆ | X₁₆ |
| T | T172 | T172 | T172 | T180 | T180 | T180 | T168 | T181 |
| X₂ | X₂ | X₂ | X₂ | X₂ | X₂ | X₂ | X₂ | X₂ |
| H | H175 | H175 | H175 | H183 | H183 | H183 | H171 | F184 |
| R | R176 | R176 | R176 | R184 | R184 | R184 | R172 | R185 |
| L | L177 | L177 | L177 | L185 | M185 | L185 | L173 | L186 |
| X₆₄₋₇₆ | X₆₄ | X₆₄ | X₆₅ | X₆₄ | X₆₄ | X₆₄ | X₆₄ | X₇₂ |
| P | P242 | P242 | P243 | P250 | P250 | P250 | P238 | P259 |
| D/K | D243 | D243 | D244 | D251 | D251 | D251 | D239 | K260 |
| X₂ | X₂ | X₂ | X₂ | X₂ | X₂ | X₂ | X₂ | X₁ |
| R | R246 | R246 | R247 | R254 | R254 | R254 | R242 | R262 |
| X₀₋₁ | — | — | — | — | — | — | — | X₁ |
| V/L | V247 | V247 | V248 | L255 | L255 | L255 | L243 | L264 |
| X₇₂₋₇₅ | X₇₂ | X₇₂ | X₇₂ | X₇₄ | X₇₄ | X₇₄ | X₇₄ | X₇₅ |
| N | N320 | N320 | N321 | N330 | N330 | N330 | N318 | N336 |
| X₂ | X₂ | X₂ | X₂ | X₂ | X₂ | X₂ | X₂ | X₂ |
| Y | Y323 | Y323 | Y324 | Y333 | Y333 | Y333 | Y321 | Y339 |
| X₃₈₋₅₅ | X₃₈ | X₃₈ | X₃₈ | X₃₈ | X₃₈ | X₃₈ | X₃₈ | X₅₅ |
| M/L/I | M362 | M362 | M363 | L372 | I372 | I372 | I360 | I395 |
| -X₀₋₁-P | P363 | P363 | P364 | P373 | X₁-P374 | X₁-P374 | X₁-P362 | X₁-P397 |
| M/I/V | M364 | M364 | M365 | I374 | V375 | V375 | V363 | V398 |
| X₂ | X₂ | X₂ | X₂ | X₂ | X₂ | X₂ | X₂ | X₂ |
| R | R365 | R365 | R365 | R365 | R365 | R365 | R365 | R365 |

The conserved portion of the p clamp consensus protein of SEQ ID NO: 9 does not start until approximately one-third the distance from the N-terminus of the full length β clamp protein. Why this is the case is apparent from the peptide-β structures. Each β half clamp (i.e. monomer) consists of three globular domains. The peptides bind to a region that involves the C-terminal and middle domains (domains 2 and 3), but do not involve interaction with the N-terminal domain (domain 1). Hence, the peptide binding motif of the full length β clamp protein starts some distance from the N-terminus.

The 1.6 angstrom high resolution structure of compounds bound to the β clamp reveals detailed backbone interactions involved in this crucial chemical-clamp connection. The compound interacts with V247, P242, R152, R246, M362, T172. These amino acid side chains are conserved among Gram positive and Gram negative bacteria. This explains how some compounds are able to inhibit growth of both Gram positive and Gram negative bacteria.

The peptide which is capable of binding to a portion of the β clamp protein can be further defined with respect to the β clamps for specific bacteria.

In the case of Escherichia coli, the peptide which is capable of binding to a portion of the β clamp protein is capable of binding to one or more of the following residues of the Escherichia coli β clamp protein (SEQ ID NO: 1): R152, L155, T172, H175, R176, L177, P242, D243, R246, V247, N320, Y323, M362, P363, M364, and M365. For example, a suitable peptide capable binding to a portion of the β clamp protein is RLLNDLRGLIGSEQVELEFD (SEQ ID NO: 10).

For Pseudomonas aeruginosa, the peptide which is capable of binding to a portion of the β clamp protein is capable of binding to one or more of the following residues of the Pseudomonas aeruginosa β clamp protein (SEQ ID NO: 2): R152, L155, T172, H175, R176, L177, P243, D244, R247, V248, N321, Y324, M363, P364, M365, and R366. For example, a suitable peptide capable of binding to a portion of the β clamp protein is DLIQALRDQFGRDNVFLNYR (SEQ ID NO: 11).

In the case of Salmonella typhimurium, the peptide which is capable of binding to a portion of the β clamp protein is capable of binding to one or more of the following residues of the Salmonella typhimurium β clamp protein (SEQ ID NO: 3): R152 L155, T172, H175, R176, L177, P250, D242, D243, R246, V247, N320, Y323, M362, P363, M364, and R365. For example, a suitable peptide capable binding to a portion of the β clamp protein is RLLNDLRGLIGSEQVELEFD (SEQ ID NO: 12).

For Staphylococcus aureus, the peptide which is capable of binding to a portion of the β clamp protein is capable of binding to one or more of the following residues of the Staphylococcus aureus β clamp protein (SEQ ID NO: 4): R160, L163, T180, H183, R184, L185, P250, D251, R254, L255, N330, Y333, L372, P373, I374 and R375. For example, a suitable peptide capable binding to a portion of the β clamp protein is DELGSLPNLPDKAQLSIFDM (SEQ ID NO: 13).

In the case of Streptococcus pneumoniae, the peptide which is capable of binding to a portion of the β clamp protein is capable of binding to one or more of the following residues of the Streptococcus pneumoniae β clamp protein (SEQ ID NO: 5): R159, L162, T180, H183, R184, M185, P250, D254, L255, N330, Y333, I372, P374, V375, and R376. For example, a suitable peptide capable binding to a portion of the β clamp protein is MGILGNMPEDNQLSLFDELF (SEQ ID NO: 14).

In the case of Streptococcus pyogenes, the peptide which is capable of binding to a portion of the β clamp protein is capable of binding to one or more of the following residues of the Streptococcus pyogenes β clamp protein (SEQ ID NO: 6): R159, L162, T180, H183, R184, M185, P250, D251, R254, L255, N330, Y333, I372, P374, V375, and R376. For example, a suitable peptide capable binding to a portion of the β clamp protein is DEMGILGNMPEDNQLSLFDDFF (SEQ ID NO: 15).

For Enterococcus faecalis, the peptide which is capable of binding to a portion of the β clamp protein is capable of binding to one or more of the following residues of the Enterococcus faecalis β clamp protein (SEQ ID NO: 7): R148, L151, T168, H171, R172, L173, P238, D239, R242, L243, N318, Y321, I360, P362, V363, and R364. For example, a suitable peptide capable binding to a portion of the β clamp protein is ENGVLKDLPDENQLSLFDML (SEQ ID NO: 16).

In the case of Mycobacterium tuberculosis, the peptide which is capable of binding to a portion of the β clamp protein is capable of binding to one or more of the following residues of the Mycobacterium tuberculosis β clamp protein (SEQ ID NO: 8): L161, L164, T181, F184, R185, L186, P259, K260, R262, L264, N336, Y339, I395, P397, V398, and R399. For example, a suitable peptide capable binding to a portion of the β clamp protein is PSPALMGDLKELLGPGCLGS (SEQ ID NO: 17).

Although the binding pocket consensus sequence in the beta clamp is highly conserved, there are slight differences in the amino acid residues which may be used to develop a narrow spectrum antibiotic. A narrow spectrum antibiotic could be useful in some cases, as they may result in producing fewer side effects and may also help reduce the possibility of generating drug resistant bacteria. For example, a compound that binds specifically with the residues L161, K260, and/or F184 in Mycobacterium tuberculosis, which differ from the consensus, may exhibit antibiotic activity specific for this bacteria rather than being a broad spectrum antibiotic. In addition, a compound that binds to the M185 residue specific to Streptococcus pyogenes may provide a narrow spectrum antibiotic for this organism. As another example, the V247,248 in E. coli, Salmonella typhimurium, and Pseudomonas aeruginosa is occupied by a leucine residue in other bacteria, and a compound that interacts specifically with the valine may yield a narrow spectrum antibiotic for these gram negative organisms.

In carrying out the method of the present invention, the determining step can be carried out by evaluating whether the candidate antibacterial compound prevents binding between the a clamp protein and the peptide which is capable of binding to a portion of the β clamp protein. Alternatively, such determining can be achieved by evaluating whether the candidate antibacterial compound displaces the peptide which is capable of binding to a portion of the β clamp protein from the β clamp protein.

In one embodiment of the present invention, the above-described method is carried out where the contacting step involves contacting the β clamp protein and the second peptide in the absence of the test compound to form a binding complex and contacting the binding complex with the test compound.

The method of the present invention can additionally include determining the polymerase activity of the candidate compound in an in vitro polymerase activity assay. Alternatively, the polymerase activity of the candidate compound can be determined in an in vitro bacterial growth assay.

In another aspect of the present invention, the subject method can be followed by contacting a Gram negative and a Gram positive bacterium with the candidate compound and determining the ability of the candidate compound to modulate growth of the Gram negative bacterium and the Gram positive bacterium. If the candidate compound inhibits growth of the Gram positive bacterium and does not substantially inhibit growth of the Gram negative bacterium, then the candidate compound is a Gram positive-specific bacterial growth inhibitor. If the candidate compound inhibits growth of the Gram negative bacterium and does not substantially inhibit growth of the Gram positive bacterium, then the candidate compound is a Gram negative-specific bacterial growth inhibitor.

The replication protein compositions used to identify these pharmacological agents are in partially pure or completely pure form and are typically recombinantly produced. The replication protein may be part of a fusion product with another peptide or polypeptide (e.g., a polypeptide that is capable of providing or enhancing protein-protein binding, stability under assay conditions (e.g., a tag for detection or anchoring, etc.). The assay mixtures comprise a natural intracellular replication protein binding target, such as beta protein and polymerase, or a peptide that binds beta. For binding assays, while native binding targets may be used, it is frequently preferred to use portions (e.g., peptides) thereof so long as the portion provides binding affinity and avidity to the subject replication protein (beta) conveniently measurable in the assay. The assay mixture also comprises a candidate pharmacological agent. Generally, a plurality of assay mixtures are run in parallel with different agents to obtain a response to various chemical structures. Typically, one of these serves as a negative control (i.e. at zero chemicals or below the limits of assay detection). Additional controls are often present such as a positive control, a dose response curve, use of known inhibitors, use of control heterologous proteins, etc. Candidate agents encompass numerous chemical classes, though typically they are organic compounds; preferably they are small organic compounds and are obtained from a wide variety of sources, including libraries of synthetic or natural compounds. A variety of other reagents may also be included in the mixture. These include reagents like salts, buffers, neutral proteins (e.g., albumin, detergents, etc.), which may be used to facilitate optimal binding and/or reduce nonspecific or background interactions, etc. Also, reagents that otherwise improve the efficiency of the assay (e.g., protease inhibitors, nuclease inhibitors, antimicrobial agents, etc.) may be used.

The present invention provides an assay used to discover chemical compounds with antibiotic activity. One embodiment of the present invention uses fluorescent rhodamine labeled peptides corresponding to the C-terminal residues of E. coli Pol III α subunit, and S. pyogenes Pol C and S. aureus Pol C. The peptide is small and thus rotates rapidly in solution, giving it a low rotation anisotropy of fluorescent light emission. When the small peptide binds the large β clamp (approximately 80 kDa), it rotates in solution much slower due to the larger size it is associated with. This gives a higher fluorescent anisotropy compared to the peptide in solution.

A mixture of β and fluorescent polymerase C-tail peptide are combined at concentrations that result in most of the fluorescent peptide being bound to β, thus giving a high anisotropy value. A chemical compound that binds to β will displace the peptide, and the rotational anisotropy will decrease. After incubation with a compound, the presence or absence of activity, or specific binding between the replication protein and one or more binding targets, is detected by fluorescence anisotropy.

Several methods could conceivably be used to monitor β interaction with polymerase or any peptide that competes with the polymerase for binding β. The monitoring method could be used in a displacement assay with compounds to achieve the same end as the peptide displacement assay described above. Examples of assays that monitor binding between two components, like the β and α peptide include, Biacore Surface Plasmon Resonance, ELISA antibody based assays, proximity assays using FRET, antibody pull down assays, etc. Further, other peptides or proteins that compete with polymerase or bind the same site on β could be used in the assay. For example, DNA ligase, Pol I, MutS, UmuCD and Pol IV bind to β, and these proteins, or peptides derived from them, could be used. Alternatively, phage display or other techniques could identify peptides that bind β, and, if they bind the same spot as polymerase, they could find use in the assay. In addition to peptides and proteins, any type of molecule that binds to β in the same or overlapping place on β as polymerase may find utility in a screen displacement type assay to identify physiologically active compounds. The resultant mixture of β clamp and its interacting partner (e.g. peptide) is incubated under conditions whereby, but for the presence of the candidate pharmacological agent, the replication protein binds the interacting partner. The mixture of components can be added in any order that provides for the requisite bindings. Incubations may be performed at any temperature which facilitates binding, typically between 4 and 40° C., more commonly between 15° and 40° C. Incubation periods are likewise selected for binding but also minimized to facilitate rapid, high-throughput screening, and are typically between 0.1 and 10 hours, preferably less than 5 hours, more preferably less than 2 hours.

An embodiment of the present invention uses peptides and demonstrates that peptides derived from bacterial replicases bind β clamps. It was originally believed that only an internal sequence of E. coli Pol III alpha subunit bind β (Kim, D. R. et al., “Identification of the β-binding Domain of the a Subunit of Escherichia coli Polymerase III Holoenzyme,” J. Biol. Chem., 271:20699-20704, (1996); Dalrymple, B. P., et. al., “A Universal Protein-Protein Interaction Motif in the Eubacterial DNA Replication and Repair Systems,” Proc. Natl. Acad. Sci., USA., 98:11627-11632, (2001), which are hereby incorporated by reference in their entirety). However, the present invention shows that the C-terminus of E. coli Pol III also binds the clamp. This is true both for Gram negative E. coli Pol III and Gram positive S. pyogenes and S. aureus Pol C. The S. pyogenes and S. aureus Pol C peptides bind both E. coli β and S. pyogenes β. Furthermore, the C-terminal peptides of E. coli alpha, S. pyogenes Pol C and S. aureus Pol C inhibit DNA synthesis by the E. coli DNA polymerase III replicase. In fact, these same three peptides also inhibit DNA synthesis by the S. pyogenes PolC holoenzyme and by the S. aureus Pol C holoenzyme. These results can be explained by an interaction between the clamp and the polymerase that has been conserved during the evolutionary divergence of Gram positive and Gram negative cells. An inhibitor that would disrupt this interaction would be predicted to have a broad spectrum of antibiotic activity, shutting down replication in Gram negative and Gram positive cells alike. This assay, and others based on this interaction, can be devised to screen compounds for such inhibition. Further, since the proteins in this assay are highly overexpressed through recombinant techniques, sufficient quantities of the protein reagents can be obtained for screening hundreds of thousands of compounds.

Compounds were screened for ability to displace fluorescent peptide from the beta clamp. Rotational anisotropy was used to detect displacement in this particular example. Compound hits were also examined for their potency in the assay by titrating them into the peptide displacement assay. Inhibition results indicate that compounds bind to β with affinities that range from 1-100 μM. Highly accurate K_{d} values for compounds binding to β could be determined using various common techniques, for example, fluorescence, nmr and itc (i.e. isothermal calorimetry).

Different compounds that scored positive in the peptide displacement assay were then examined for their ability to inhibit DNA replication of the polymerase-β interaction using the polymerase, the clamp, and the clamp loader in a polymerase holoenzyme DNA synthesis assay (see Studwell et al., “Processive Replication is Contingent on the Exonuclease Subunit of DNA Polymerase III Holoenzyme,” J. Biol. Chem. 265(2):1171-1178 (1990), which is hereby incorporated by reference in its entirety). The assay makes use of the fact that the β clamp provides the polymerase with high processivity. The substrate is a large 7.2 kb M13mp18 ssDNA genome which is primed with only a single DNA oligonucleotide primer. The conditions are such that DNA synthesis is almost entirely dependent on the β clamp. These conditions include the addition of the DNA polymerase, the beta clamp, the clamp loader complex, and ATP which are needed to open and close the β clamp onto DNA.

Not all of the compounds that displace polymerase peptide from β were functional in this replication assay. Those that were able to inhibit replication synthesis were examined further. The compound hits were titrated into the reaction at different concentrations to obtain the IC₅₀. IC₅₀ values were also obtained using the rhodamine peptide displacement assay.

Compounds that tested positive in these assays using protein derived from Gram negative bacteria were also tested in S. pyogenes replication and peptide displacement assays. A subset of the compounds also tested positive in these assays. Compounds were also tested for whether they inhibited yeast Pol delta replicase. Most compounds that inhibited bacterial replicases did not inhibit the eukaryotic Pol delta replicase, or Pol I. Therefore, testing in yeast eliminates antibacterial compounds that might also function in a eukaryotic cell.

The present invention demonstrates that some of compounds inhibit bacterial cell growth. Many compounds inhibited the growth of Gram positive and/or Gram negative cells. Further, the compounds do not inhibit a eukaryotic cell. Therefore, the present invention demonstrates a method of identifying compounds that modulate bacterial cell growth.

Crystal structures of E. coli Pol III α peptide bound to E. coli β, and a compound bound to β were solved to determine whether they truly bind the same spot on the β clamp. The present invention demonstrates that they both bind at an overlapping space on β clamp and involve interaction with some of the same conserved residues. S. aureus and S. pyogenes Pol C peptides bound to E. coli β were also cocrystalized, and they bind the same site as E. coli Pol III peptide. It is also possible that the structure of the compound binding site in the β clamp disclosed herein could be used to identify binding compounds by computational modeling methods. Compounds identified by modeling would be obtained or synthesized and then tested for their ability to displace peptides from the β clamp.

The present invention provides methods by which replication proteins from Gram positive and Gram negative bacteria are used to discover new pharmaceutical agents. The function of replication proteins is quantified in the presence of different compounds. A compound that inhibits the function is a candidate antibiotic. Some replication proteins from a Gram positive bacteria and from a Gram negative bacteria can be interchanged for one another. Hence, they can function as mixtures. Reactions that assay for the function of enzyme mixtures consisting of proteins from Gram positive bacteria and from Gram negative bacteria can also be used to discover drugs. Suitable E. coli replication proteins are the subunits of its Pol III holoenzyme which are described in U.S. Pat. Nos. 5,583,026 and 5,668,004, which are hereby incorporated by reference in their entirety.

The methods described here demonstrating activity behavior of S. aureus, S. pyogenes, and E. coli are likely to generalize to all members of Gram positive and Gram negative bacteria.

The present invention describes a method to identify compounds that inhibit the ability of a beta subunit and a DNA polymerase, or other interacting partner, to interact physically. This method involves contacting the beta subunit with molecule that binds to β at the same site as the DNA polymerase in the presence of the candidate pharmaceutical to form a reaction mixture. The reaction mixture is subjected to conditions under which the β binding component and the beta subunit interact in the absence of the candidate pharmaceutical. The reaction mixture is then analyzed for interaction between the beta and the beta binding unit (such as DNA polymerase or polymerase peptide). The candidate pharmaceutical is detected by the absence of interaction between beta and the β binding unit.

### EXAMPLES

### Example 1

### Materials

Labeled deoxy- and ribonucleoside triphosphates were from Dupont—New England Nuclear; unlabelled deoxy- and ribonucleoside triphosphates were from Pharmacia—LKB; E. coli replication proteins were purified as described, alpha, epsilon, gamma, and tau (Studwell, et al., “Processive Replication is Contingent on the Exonuclease Subunit of DNA Polymerase III Holoenzyme,” J. Biol. Chem., 265:1171-1178 (1990), which is hereby incorporated by reference in its entirety), beta (Kong, et. al, “Three Dimensional Structure of the Beta Subunit of Escherichia coli DNA Polymerase III Holoenzyme: A Sliding DNA Clamp,” Cell, 69:425-437 (1992), which is hereby incorporated by reference in its entirety), delta and delta prime (Dong, et. al., “DNA Polymerase III Accessory Proteins. I. HolA and holB Encoding δ and δ′, J. Biol. Chem., 268:11758-11765 (1993), which is hereby incorporated by reference in its entirety), chi and psi (Xiao, et. al., “DNA Polymerase III Accessory Proteins. III. HolC and holD Encoding chi and psi,” J. Biol. Chem., 268:11773-11778 (1993), which is hereby incorporated by reference in its entirety), theta (Studwell-Vaughan, et al., “DNA Polymerase III Accessory Proteins. V. Theta Encoded by holE,” J. Biol. Chem., 268:11785-11791 (1993), which is hereby incorporated by reference in its entirety), and SSB (Weiner, et. al., “The Deoxyribonucleic Acid Unwinding Protein of Escherichia coli,” J. Biol. Chem., 250:1972-1980 (1975), which is hereby incorporated by reference in its entirety). E. coli Pol III core, and gamma complex (composed of subunits: gamma, delta, delta prime, chi, and psi) were reconstituted as described in Onrust, et. al., “Assembly of a Chromosomal Replication Machine: Two DNA Polymerases, a Clamp Loader and Sliding Clamps in One Holoenzyme Particle. I. Organization of the Clamp Loader,” J. Biol. Chem., 270:13348-13357 (1995), which is hereby incorporated by reference in its entirety. Pol III* was reconstituted and purified as described in Onrust, et. al., “Assembly of a Chromosomal Replication Machine: Two DNA Polymerases, a Clamp Loader and Sliding Clamps in One Holoenzyme Particle. III. Interface Between Two Polymerases and the Clamp Loader,” J. Biol. Chem., 270:13366-13377 (1995), which is hereby incorporated by reference in its entirety. Staphylococcus aureus Pol C and β were purified as described (Klemperer, N., et. al., “Cross Utilization of the Beta Sliding Clamp by Replicative Polymerases of Evolutionary Divergent Organisms,” J. Biol. Chem., 275:26136-26143 (2000), which is hereby incorporated by reference in its entirety). Streptococcus pyogenes Pol C, β, SSB, δ, δ′, and τ were purified as described (Bruck I., et al., “The DNA Replication Machine of a Gram-Positive Organism,” J Biol Chem., 275:28971-28983, (2000), which is hereby incorporated by reference in its entirety). The S. pyogenes ατ and δδ′ complexes were reconstituted and purified from unbound subunits, as described (Bruck I., et al., “The DNA Replication Machine of a Gram-Positive Organism.” J Biol Chem., 275:28971-28983, (2000) which is hereby incorporated by reference in its entirety). M13mp18 ssDNA was isolated and primed with a DNA oligonucleotide as described (Turner, J., et al., “Cycling of Escherichia coli DNA Polymerase III From One Sliding Clamp to Another: Model for Lagging Strand,” Methods in Enzymol., 262:442-449, (1995), which is hereby incorporated by reference in its entirety). Protein concentrations were quantitated by the Protein Assay (Bio-Rad) method using bovine serum albumin (BSA) as a standard. DNA oligonucleotides were synthesized by Oligos etc. Calf thymus DNA was from Sigma. Buffer A is 20 mM Tris-HCl (pH=7.5), 0.5 mM EDTA, 2 mM DTT, and 20% glycerol. Replication buffer was 20 mM Tris-Cl (pH 7.5), 8 mM MgCl₂, 5 mM DTT, 0.5 mM EDTA, 40 μg/ml BSA, 4% glycerol, 0.5 mM ATP, 3 mM each dCTP, dGTP, dATP, and 20 μM [α-³²P]dTTP.

### Example 2

### An Assay for Binding of the C-Terminal Residues of Bacterial Pol C (Pol III) Polymerases to the β Camp

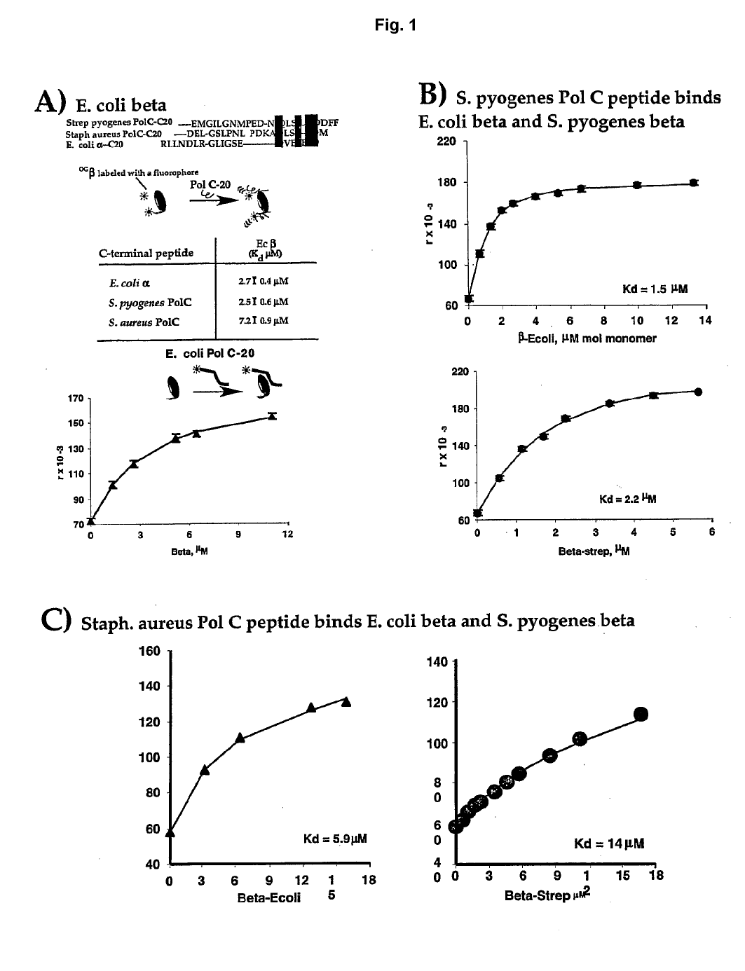

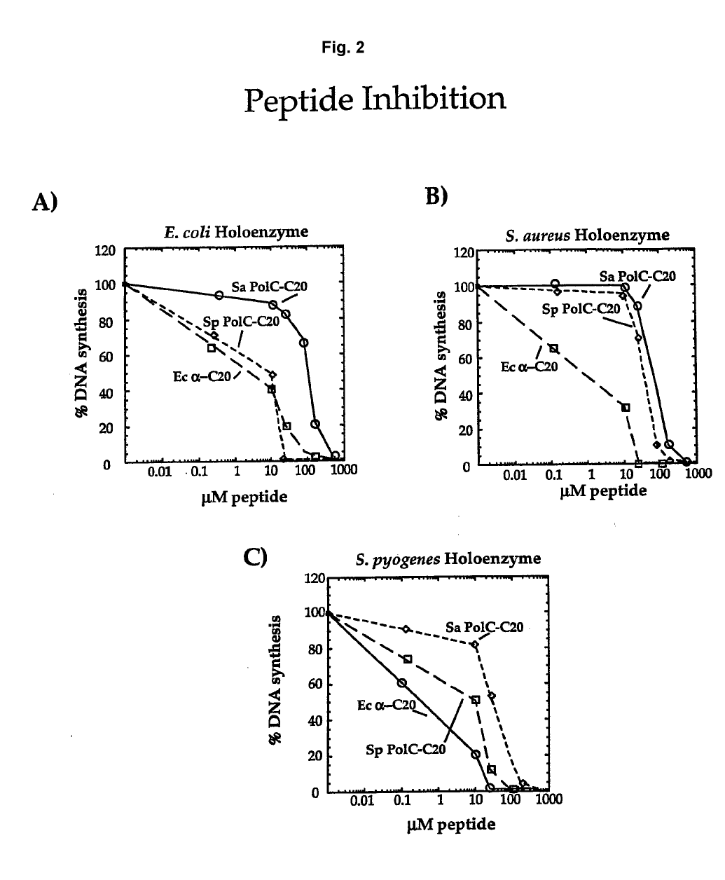

A simple and quantitative assay has been developed to monitor binding of a E. coli Pol III peptide to E. coli beta (López de Saro, F. J., et. al., “Competitive Processivity—Clamp Usage by DNA Polymerase During DNA Replication and Repair,” EMBO J., 22:6408-6418 (2003), which is hereby incorporated by reference in its entirety). A rhodamine-labeled 20 mer peptide corresponds to the sequence of the C-terminal 20 residues of E. coli Pol III α subunit. Titration of the beta clamp into the rhodamine labeled peptide resulted in a fluorescence increase of the rhodamine labeled peptide upon binding to the beta molecule. A plot of the fluorescence change with beta concentration provides a K_{d} value for the interaction between these molecules (FIG. 1A).

This assay has also been examined using Streptococcus pyogenes β and rhodamine labeled peptides corresponding to C-terminal residues of Streptococcus pyogenes Pol C and E. coli Pol III. The results showed that these Gram positive and Gram negative DNA polymerase derived peptide sequences bound to S. pyogenes β (FIG. 1B). The C-terminal peptide corresponding to Staphylococcus aureus Pol C was also tested for binding to these β clamps (FIG. 1C). The result showed that the S. aureus Pol C peptide bound to the Gram positive S. pyogenes β clamp and to the Gram-negative β clamp. These results indicated that the key polymerase binding residues in the hydrophobic pocket of β clamps of Gram positive and Gram negative bacteria are highly conserved.

### Example 3

### Polymerase Peptides Inhibit Bacterial Replicases

Assays were performed using primed M13mp18 ssDNA coated with SSB as substrate. Each reaction was 25 μl and contained 72 ng primed M13mp18 ssDNA, 1 μg SSB, 0.1 mM MgCl₂, 20 mM TrisHCl (pH 7.5), 0.1 mM EDTA, 5 mM DTT, 40 μg/ml BSA, 4% glycerol, 0.5 mM EDTA, 2 mM ATP, 60 μM each of dCTP, dATP, dGTP and 20 μM α³²-P dTTP. Replicases consisted of the following for each E. coli, S. pyogenes, and S. aureus: 50 ng Pol III core (E. coli) or Pol C (S. pyogenes and S. aureus), 200 ng τ, 20 ng δδ′ and 40 ng β. Peptides, when present, were added to the indicated concentration. Reactions were assembled on ice in the absence of the polymerase, then shifted to 37° C. for 3 min before initiating synthesis by addition of the polymerase. Reactions were incubated a further 3 min prior to quenching and quantitation of synthesis.

### Example 4

### A Peptide Displacement Assay Screen for Antibacterial Compound Inhibitors

The peptide binding results presented in Example 2 demonstrated the high conservation of key polymerase binding residues in the hydrophobic pocket of beta clamps. A compound that disrupts this important interaction may be expected to inhibit the central replication reaction of bacteria, which requires the polymerase-beta connection. To develop a screen for disruption of this interaction, a mixture of rhodamine-labeled peptide and beta was made, resulting in most of the rhodamine-labeled peptide being bound to beta. A compound that disrupts this interaction should displace the rhodamine-labeled peptide from beta and produce a change in rotational anisotropy.

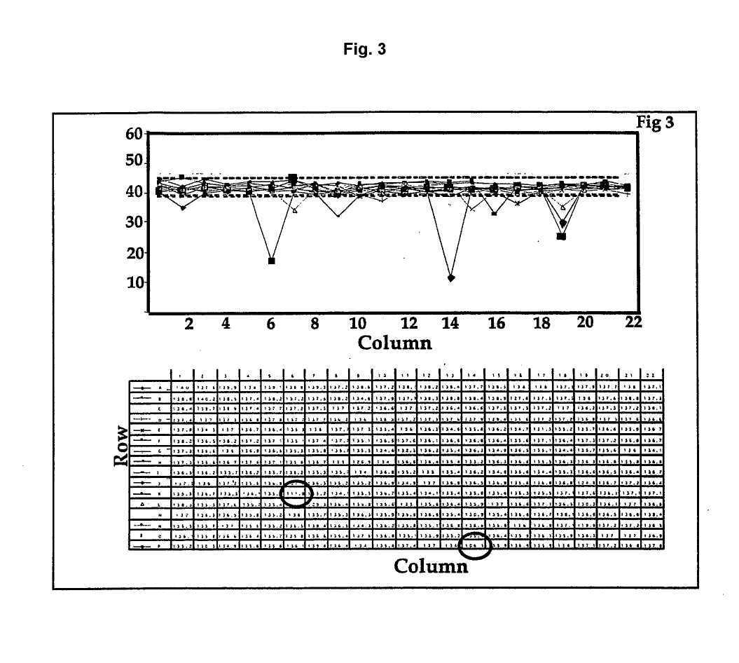

This assay was then used to screen compounds. Into each well was placed 0.2 μl compound at 5 mM in DMSO. Then 15 μl of the rhodamine peptide-β complex was added. This reaction mixture contained 6 μM E. coli β (as monomer), 1 μM rhodamine Pol III peptide, 20 mM Tris-HCl (ph 7.5), 5 mM DTT, and 0.5 mM EDTA. Control wells lacking compounds included rhodamine labeled peptide with no β clamp, rhodamine peptide-β complex, and rhodamine peptide-β complex to which different concentrations of unlabeled peptide competititor was added. Fluorescence anisotropy was then measured using a plate reader. Typical results are illustrated in FIG. 3. Results from this assay were highly stable even after sitting for 24 hours at room temperature. Presumptive hits were cherry-picked and retested in duplicate. Compound structures of reconfirmed hits were examined and sorted into groups having common template structures.

### Example 5

### IC50 Titrations

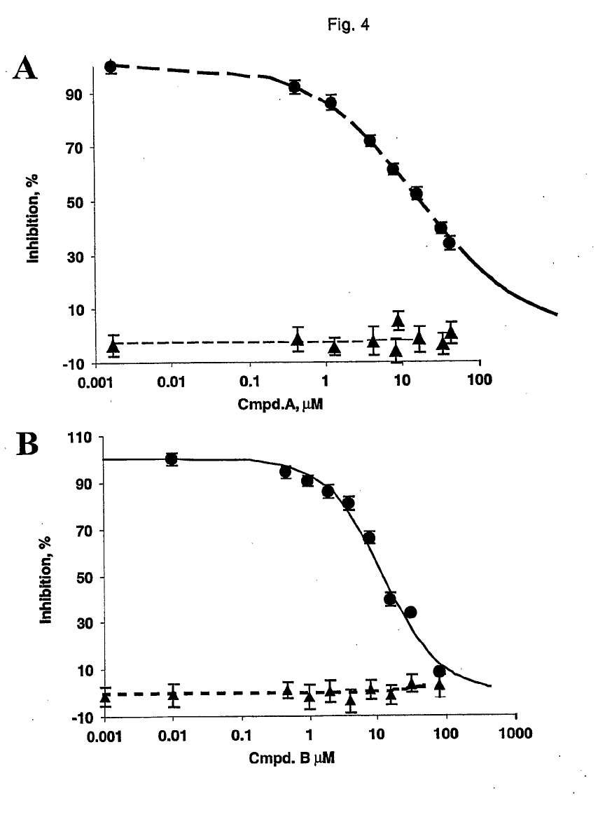

Titrations of compounds that had tested positive for peptide displacement were titrated into reactions that contained 6 μM E. coli β, 1 μM rhodamine labeled E. coli Pol III α peptide, 20 mM TrisCl (pH 7.5), 5 mM DTT, and 0.5 mM EDTA. Fluorescence anisotropy data was collected on a PTI spectroflorimeter. Examples of IC50 titrations were shown in FIG. 4.

### Example 6

### A Subset of Compounds that Test Positive for Peptide Displacement also Inhibit the E. coli Pol III Holoenzyme in vitro

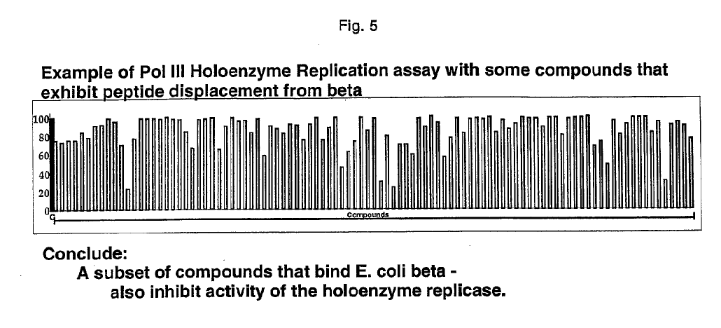

Reactions (25 μl) contained 72 ng primed M13mp18 ssDNA, 0.8 μg SSB, 10 ng β, 50 ng Pol III*, 40 μM compound and 20 mM Tris HCl (pH 7.5), 0.1 mM EDTA, 5 mM DTT, 4% glycerol, 40 mg/ml BSA, 0.5 mM ATP, 60 μM each dCTP, dGTP, dATP and dTTP. Reactions were allowed to proceed 10 min at room temperature, then stopped upon adding 75 μl 166 mM EDTA, 0.33 mM Tris HCl (pH 7.5) and 0.4 μl Picogreen dye, followed by reading fluorescence on a plate reader. An example of results from the testing of some compounds that tested positive for peptide displacement are shown in FIG. 5.

### Example 7

### A Subset of Compounds Selected Using the E. coli β Clamp can also Displace S. pyogenes Pol C Peptide from the S. pyogenes β Clamp

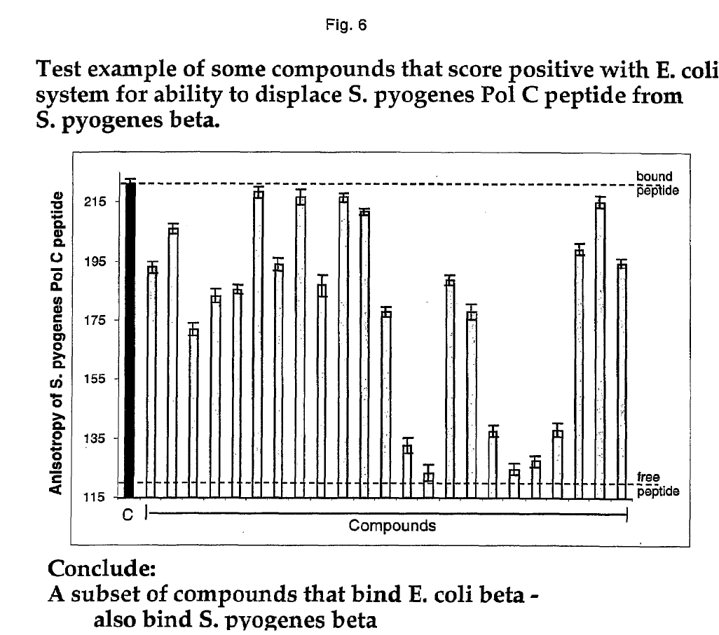

Compounds that displace E. coli Pol III peptide from E. coli β were tested for ability to displace S. pyogenes Pol C peptide from S. pyogenes β. Reaction mixtures contained 1 μM S. pyogenes rhodamine labeled Pol C peptide and 3 μM S. pyogenes β in 20 mM Tris-HCl (pH 7.5), 5 mM DDT and 0.5 mM EDTA. An example of some of the results is shown in FIG. 6, and they demonstrates that some compounds functioned in both systems.

### Example 8

### Holoenzyme Replication Assays

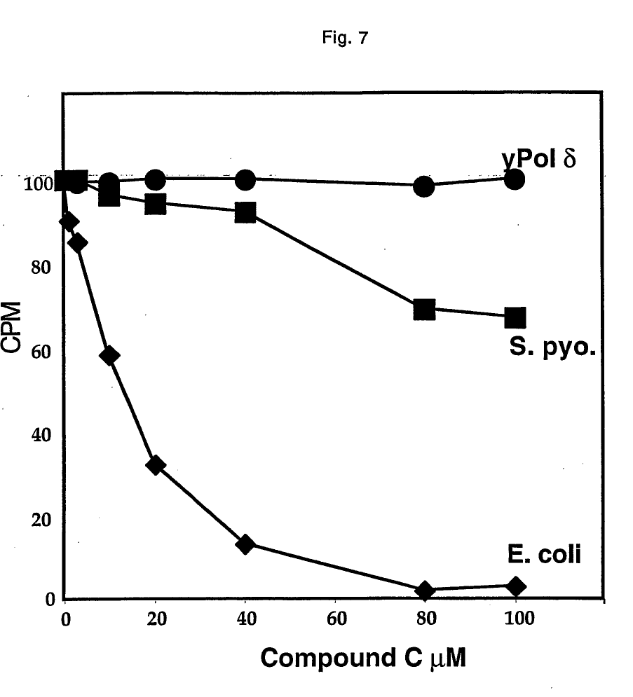

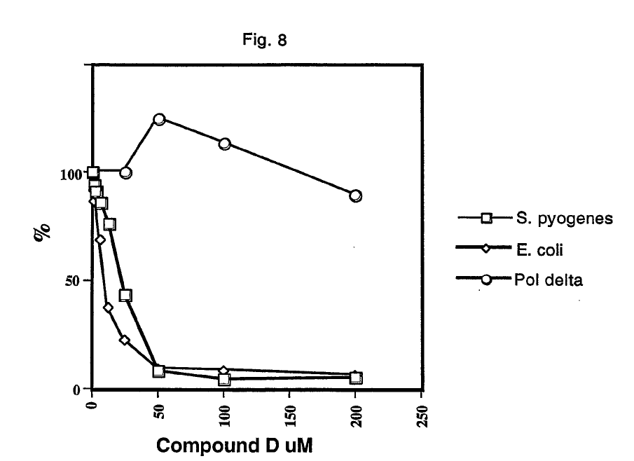

Assays were performed using primed M13mp18 ssDNA coated with SSB as substrate. Each time point was 25 μl containing 72 ng primed M13mp18 ssDNA, 0.8 μg, SSB, 0.1 MM MgCl₂, 20 mM TrisHCl (pH 7.5), 0.1 mM EDTA, 5 mM DTT, 40 μg/ml BSA, 4% glycerol, 0.5 mM EDTA, 1 mM ATP, 60 μM each of dCTP, dATP, dGTP and 20 μM α³²-P dTTP. For E. coli, each 25 μl contained 2.5 ng β, 10 ng γ complex, 10 ng Pol III core. Reactions were incubated for 10 min at room temperature. For S. pyogenes, each 25 μl contained 20 ng β, 10 ng Pol C-τ, and 10 ng δδ′. Reactions were incubated 2.5 min at room temperature. For yeast Pol δ, reactions contained 25 ng RFC, 4 ng Pol δ and 7 ng PCNA. Reactions were incubated 10 min at 30° C. Results for two different compounds are shown in FIGS. 7 and 8.

### Example 9

### Some Compounds Inhibit Cell Growth

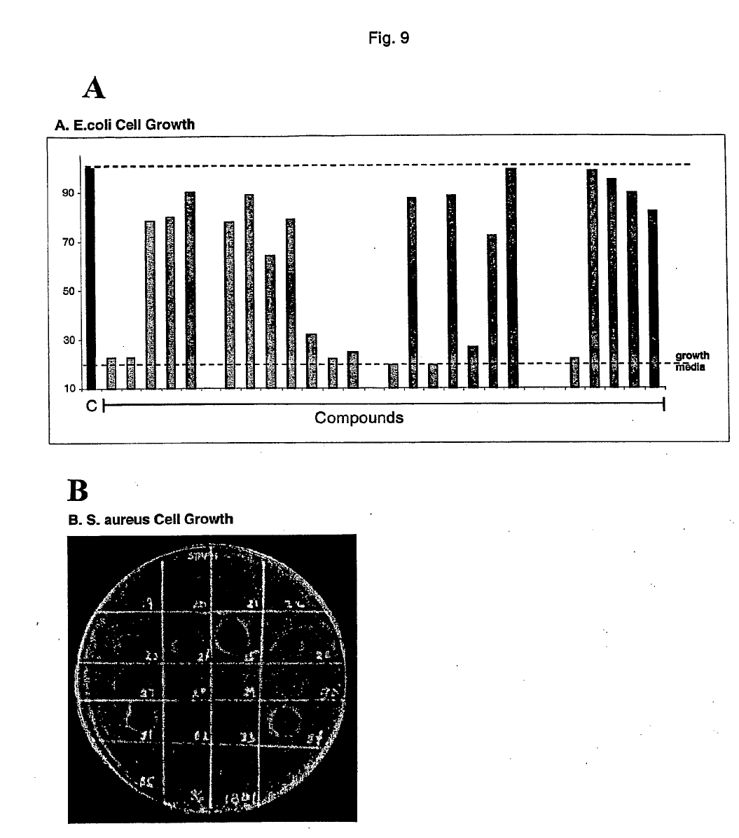

E. coli cell growth in the presence of peptide displacing compounds was tested using 33 μM compound in a final volume of 25 μl containing bacteria diluted in LB from an overnight culture to an OD of 0.05. Plates were shaken while incubated at 37° C., then were read on a plate reader for cell growth. Results that were obtained are in FIG. 9A. S. aureus cell growth inhibition was tested on an LB plate by spotting 0.5 μl of 5 mM compound on a lawn of cells. An example of a result from this assay is shown in FIG. 9B.

These compounds have also been tested for growth inhibition of eukaryotic Sacchromyces cerevisiae, but no growth inhibition was observed for this eukaryotic cell.

### Example 10

### X-ray Crystal Structure of E. coli Pol III and S. aureus Pol C Peptides Bound to β Reveal Key Conserved Clamp Residues that Bind Replicases

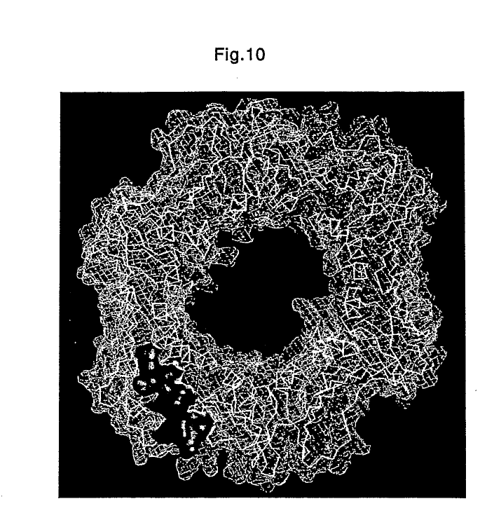

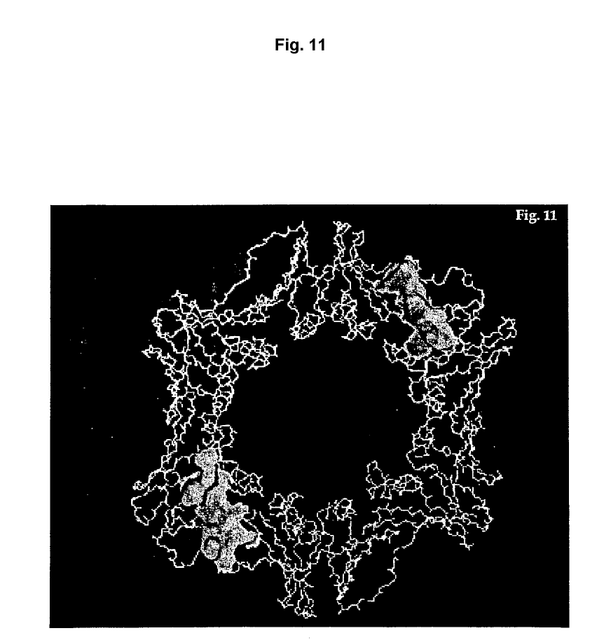

Crystals of E. coli β with E. coli Pol III peptide bound to it (FIG. 10) and with S. aureus Pol C peptide bound to it (FIG. 11) were obtained by the hanging drop method (McPherson, “Current Approaches to Macromolecular Crystalization,” Eur. J. Biochem. 189:1-23 (1990), which is hereby incorporated by reference in its entirety). Crystals were formed in 24-26% PEG, 1-3% DMSO, 0.1 M CaCl₂ and 0.1 M MES pH 6.1. Crystals were placed in an X-ray beam at Brookhaven National laboratories. Structures were solved by molecular replacement and refined to 1.90-2.03 Angstroms.

### Example 11

### Crystal Structure of Compound Bound to E. coli β

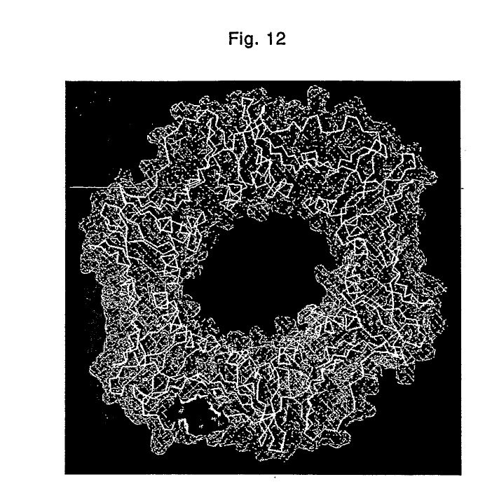

The structure of a compound bound to E. coli β was obtained using the procedure developed in Example 10. The compound binds to β (FIG. 12) in a space that overlaps the polymerase peptide binding site. The amino acid residues which form contacts with the peptides and compound include residues indicated in FIG. 13.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of identifying a candidate compound for modulating bacterial growth, the method comprising
a) providing a β clamp peptide from a bacterial replicase;
b) providing a second peptide that binds to at least one amino acid of SEQ ID NO:9 that is not designated X, wherein the second peptide does not exhibit polymerase activity;
c) providing a test compound;
d) contacting the β clamp peptide and the second peptide with the test compound;
e) determining the level of binding between the β clamp peptide and the second peptide in the presence of the test compound; and
f) comparing the level of binding between the β clamp peptide and the second peptide in the presence of the test compound to a control that does not contain the test compound, wherein a test compound that alters the level of binding between the β clamp peptide and the second peptide compared to the control is a candidate compound for modulating bacterial growth.

2. The method of claim 1, wherein the second peptide binds to at least one residue of SEQ ID NO:9 that is designated X.

3. The method of claim 1, wherein when the level of binding between the β clamp peptide and the second peptide is decreased in the presence of the test compound, the test compound is a candidate compound for modulating bacterial growth.

4. The method of claim 1, wherein, when the level of binding between the β clamp protein and the second peptide is increased in the presence of the test compound, the test compound is a candidate compound for modulating bacterial growth.

5. The method of claim 1, wherein the β clamp peptide is a full length β clamp protein.

6. The method of claim 1, wherein the β clamp peptide is derived from a Gram negative bacterium.

7. The method of claim 6, wherein the Gram negative bacterium is selected from the group consisting of Escherichia coli, Pseudomonas aeruginosa, and Salmonella typhimurium.

8. The method of claim 7, wherein the Gram negative bacterium is Escherichia coli and the second peptide binds to at least one of the following residues of an Escherichia coli β clamp protein (SEQ ID NO: 1): R152, L155, T172, H175, R176, L177, P242, D243, R246, V247, N320, Y323, M362, P363, M364, and M365.

9. The method of claim 8, wherein the second peptide comprises SEQ ID NO: 10.

10. The method of claim 7, wherein the Gram negative bacterium is Pseudomonas aeruginosa and the second peptide binds to at least one of the following residues of an Pseudomonas aeruginosa β clamp protein (SEQ ID NO:2): R152, L155, T172, H175, T176, L177, P243, D244, R247, V248, N321, Y324, M363, P364, M365, and R366.

11. The method of claim 10, wherein the second peptide comprises SEQ ID NO: 11.

12. The method of claim 7, wherein the Gram negative bacterium is Salmonella typhimurium and the second peptide binds to at least one of the following residues of a Salmonella typhimurium β clamp protein (SEQ ID NO:3): R152, L155, T172, H175, R176, L177, P250, D242, D243, R246, V247, N320, Y323, M362, P363, M364, and R365.

13. The method of claim 12, wherein the second peptide comprises SEQ ID NO: 12.

14. The method of claim 1, wherein the β clamp peptide is from a Gram positive bacterium.

15. The method of claim 14, wherein the Gram positive bacterium is selected from the group consisting of Staphylococcus aureus, Streptococcus pneumoniae, Streptococcus pyogenes, Enterococcus faecalis, and Mycobacterium tuberculosis.

16. The method of claim 15, wherein the Gram positive bacterium is Staphylococcus aureus and the second peptide binds to at least one of the following residues of a Staphylococcus aureus β clamp protein (SEQ ID NO:4): R160, L163, T180, H183, R184, L185, P250, D251, R254, L255, N330, Y333, L3762, P373, I374, and R375.

17. The method of claim 16, wherein the second peptide comprises SEQ ID NO: 13.

18. The method of claim 15, wherein the Gram positive bacterium is Streptococcus pneumoniae and the second peptide binds to at least one of the following residues of a Streptococcus pneumoniae β clamp protein (SEQ ID NO:5): R159, L162, T180, H183, R184, M185, P250, D254, L255, N330, Y333, I372, P374, V375, and R376.

19. The method of claim 18, wherein the amino acid sequence of the second peptide comprises SEQ ID NO:14.

20. The method of claim 15, wherein the Gram positive bacterium is Streptococcus pyogenes and the second peptide binds to at least one of the following residues of a Streptococcus pyogenes β clamp protein (SEQ ID NO:6): R159, L162, T180, H183, R184, M185, P250, D251, R254, L255, N330, Y333, I372, P374, V375, and R376.

21. The method of claim 20, wherein the amino acid sequence of the second peptide comprises SEQ ID NO: 15.

22. The method according to claim 15, wherein the Gram positive bacterium is Enterococcus faecalis and the second peptide binds to at least one of the following residues of an Enterococcus faecalis β clamp protein (SEQ ID NO: 7): R148, L151, T168, H171, R172, L173, P238, D239, R242, L243, N318, Y321, I360, P362, V363, and R364.

23. The method of claim 22, wherein the amino acid sequence of the second peptide comprises SEQ ID NO: 16.

24. The method of claim 15, wherein the Gram positive bacterium is Mycobacterium tuberculosis and the second peptide binds to at least one of the following residues of a Mycobacterium tuberculosis β clamp protein (SEQ ID NO:8): L161, L164, T181, F184, R185, L186, P259, K260, R262, L264, N336, Y339, I395, P397, V398, and R399.

25. The method of claim 24, wherein the amino acid sequence of the second peptide comprises SEQ ID NO: 17.

26. The method of claim 1, wherein said comparing determines whether the test compound inhibits binding between the β clamp peptide and the second peptide.

27. The method of claim 26, wherein said comparing determines whether the test compound promotes binding between the β clamp peptide and the second peptide.

28. The method of claim 1, wherein said contacting comprises:
contacting the β clamp peptide and the second peptide in the absence of the test compound, thereby forming a binding complex and
contacting the binding complex with the test compound.

29. The method of claim 1 further comprising:
determining the polymerase activity of the candidate compound in an in vitro polymerase activity assay.

30. The method of claim 1 further comprising:
determining the polymerase activity of the candidate compound in an in vitro bacterial growth assay.

31. The method of claim 1 further comprising:
contacting a Gram negative and a Gram positive bacterium with the candidate compound and
determining the ability of the candidate compound to modulate growth of the Gram negative bacterium and the Gram positive bacterium.

32. The method of claim 31, wherein if the candidate compound inhibits growth of the Gram positive bacterium and does not substantially inhibit growth of the Gram negative bacterium, then the candidate compound is a Gram positive-specific bacterial growth inhibitor.

33. The method of claim 31, wherein if the candidate compound inhibits growth of the Gram negative bacterium and does not substantially inhibit growth of the Gram positive bacterium, then the candidate compound is a Gram negative-specific bacterial growth inhibitor.


SEQUENCE LISTING (50 sequences)

SEQ ID NO: 1 - protein, 366 aa, Escherichia coli
MKFTVEREHLLKPLQQVSGPLGGRPTLPILGNLLLQVADGTLSLTGTDLEMEMVARVALVQPHEPGATTVPARKFFDICRGLPEGAEIAVQLEGERMLVRSGRSRFSLSTLPAADFPNLDDWQSEVEFTLPQATMKRLIEATQFSMAHQDVRYYLNGMLFETEGEELRTVATDGHRLAVCSMPIGQSLPSHSVIVPRKGVIELMRMLDGGDNPLRVQIGSNNIRAHVGDFIFTSKLVDGRFPDYRRVLPKNPDKHLEAGCDLLKQAFARAAILSNEKFRGVRLYVSENQLKITANNPEQEEAEEILDVTYSGAEMEIGFNVSYVLDVLNALKCENVRMMLTDSVSSVQIEDAASQSAAYVVMPMRL

SEQ ID NO: 2 - protein, 367 aa, Pseudomonas aeruginosa
MHFTIQREALLKPLQLVAGVVERRQTLPVLSNVLLVVEGQQLSLTGTDLEVELVGRVVLEDAAEPGEITVPARKLMDICKSLPNDVLIDIRVEEQKLLVKAGRSRFTLSTLPANDFPTVEEGPGSLNFSIAQSKLRRLIDRTSFAMAQQDVRYYLNGMLLEVNGGTLRSVATDGHRLAMCSLDAQIPSQDRHQVIVPRKGILELARLLTEQDGEVGIVLGQHHIRATTGEFTFTSKLVDGKFPDYERVLPRGGDKLVVGDRQQLREAFSRTAILSNEKYRGIRLQLSNGLLKIQANNPEQEEAEEEVQVEYNGGNLEIGFNVSYLLDVLGVIGTEQVRFILSDSNSSALVHEADNDDSAYVVMPMRL

SEQ ID NO: 3 - protein, 366 aa, Salmonella typhimurium
MKFTVEREHLLKPLQQVSGPLGGRPTLPILGNLLLQVADGTLSLTGTDLEMEMVARVTLSQPHEPGATTVPARKFFDICRGLPEGAEIAVQLEGDRMLVRSGRSRFSLSTLPAADFPNLDDWQSEVEFTLPQATMKRLIESTQFSMAHQDVRYYLNGMLFETEGSELRTVATDGHRLAVCSMPLEASLPSHSVIVPRKGVIELMRMLDGGENPLRVQIGSNNIRAHVGDFIFTSKLVDGRFPDYRRVLPKNPDKHLEAGCDILKQAFARAAILSNEKFRGVRLYVSENQLKITANNPEQEEAEEILDVSYGGTEMEIGFNVSYVLDVLNALKCETVRIMLTDSVSSVQIEDAASQSAAYVVMPMRL

SEQ ID NO: 4 - protein, 377 aa, Staphylococcus aureus
MMEFTIKRDYFITQLNDTLKAISPRTTLPILTGIKIDAKEHEVILTGSDSEISIEITIPKTVDGEDIVNISETGSVVLPGRFFVDIIKKLPGKDVKLSTNEQFQTLITSGHSEFNLSGLDPDQYPLLPQVSRDDAIQLSVKVLKNVIAQTNFAVSTSETRPVLTGVNWLIQENELICTATDSHRLAVRKLQLEDVSENKNVIIPGKALAELNKIMSDNEEDIDIFFASNQVLFKVGNVNFISRLLEGHYPDTTRLFPENYEIKLSIDNGEFYHAIDRASLLAREGGNNVIKLSTGDDVVELSSTSPEIGTVKEEVDANDVEGGSLKISFNSKYMMDALKAIDNDEVEVEFFGTMKPFILKPKGDDSVTQLILPIRTY

SEQ ID NO: 5 - protein, 378 aa, Streptococcus pneumoniae
MIHFSINKNLFLQALNTTKRAISSKNAIPILSTVKIDVTNEGITLIGSNGQISIENFISQKNEDAGLLITSLGSILLEASFFINVVSSLPDVTLDFKEIEQNQIVLTSGKSEITLKGKDSEQYPRIQEISASTPLILETKLLKKIINETAFAASTQESRPILTGVHFVLSQHKELKTVATDSHRLSQKKLTLEKNSDDFDVVIPSRSLREFSAVFTDDIETVEIFFANNQILFRSENISFYTRLLEGNYPDTDRLIPTDFNTTITFNVVNLRQSMERARLLSSATQNGTVKLEIKDGVVSAHVHSPEVGKVNEEIDTDQVTGEDLTISFNPTYLIDSLKALNSEKVTISFISAVRPFTLVPADTDEDFMQLITPVRTN

SEQ ID NO: 6 - protein, 378 aa, Streptococcus pyogenes
MIQFSINRTLFIHALNATKRAISTKNAIPILSSIKIKVTPTGVTLTGSNGQISIENTIPVSNENAGLLITSPGAILLEASFFINIISSLPDISINVKEIEQHQVVLTSGKSEITLKGKDVDQYPRLQEVSTENPLILKTKLLKSIIAETAFAASLQESRPILTGVHIVLSNHKDFKAVATDSHRMSQRLITLDNTSADFDVVIPSKSLREFSAVFTDDIETVEVFFSPSQILFRSEHISFYTRLLEGNYPDTDRLLMTEFETEVVFNTQSLRHAMERAFLISNATQNGTVKLEITQNHISAHVNSPEVGKVNEDLDIVSQSGSDLTISFNPTYLIESLKAIKSETVKIHFLSPVRPFTLTPGDEEESFIQLITPVRTN

SEQ ID NO: 7 - protein, 366 aa, Enterocccus faecalis
MQELQTVQRAISSKTTIPILTGVKITLTQEGLTLTGSNADISIETFLSVENEKANMQIESTGSIVLQARFFSEIIRRLPEETFTLEVLENKQVAITSGKANFIVNGLDADNYPHLPVVESHNQMKLPVHVLTKLINETVFAVSQHESRPILTGVHFILSDNSLLAVATDSHRLSQRVIPVEQAADHFDIVIPGKSLIELSRSLTNEEEIVEISIMENQVLFKTETMYFYSRLLEGNYPDTNRLIPSSFNTEVEFSVPSFLAAIERASLLSHEGRNNIVRLSIRPDAVVLYGNSPEIGKVEESLSYTASSGDPLDISFNPDYMKAALRAFGDMSIKVKFISAIRPFTLEPTEDGVQFIQLITPVRTN

SEQ ID NO: 8 - protein, 402 aa, Mycobacterium tuberculosis
MDAATTRVGLTDLTFRLLRESFADAVSWVAKNLPARPAVPVLSGVLLTGSDNGLTISGFDYEVSAEAQVGAEIVSPGSVLVSGRLLSDITRALPNKPVDVHVEGNRVALTCGNARFSLPTMPVEDYPTLPTLPEETGLLPAELFAEAISQVAIAAGRDDTLPMLTGIRVEILGETVVLAATDRFRLAVRELKWSASSPDIEAAVLVPAKTLAEAAKAGIGGSDVRLSLGTGPGVGKDGLLGISGNGKRSTTRLLDAEFPKFRQLLPTEHTAVATMDVAELIEAIKLVALVADRGAQVRMEFADGSVRLSAGADDVGRAEEDLVVDYAGEPLTIAFNPTYLTDGLSSLRSERVSFGFTTAGKPALLRPVSGDDRPVAGLNGNGPFPAVSTDYVYLLMPVRLPG

SEQ ID NO: 9 - protein, 260 aa, Artificial Sequence
  conserved beta clamp consensus protein
RXXLXXXXXXXXXXXXXXXXXXXXXXXXXXTXXHRLXXXXXXXXXXXXXXXXXXXXXXXXXXXXXXXXXXXXXXXXXXXXXXXXXXXXXXXXXXXXXXXXXXXXXXXXXXXXPXXXRXXXXXXXXXXXXXXXXXXXXXXXXXXXXXXXXXXXXXXXXXXXXXXXXXXXXXXXXXXXXXXXXXXXXXXXXXXXXXNXXYXXXXXXXXXXXXXXXXXXXXXXXXXXXXXXXXXXXXXXXXXXXXXXXXXXXXXXXXXPXXXR

SEQ ID NO: 10 - protein, 20 aa, Escherichia coli
RLLNDLRGLIGSEQVELEFD

SEQ ID NO: 11 - protein, 20 aa, Pseudomonas aeruginosa
DLIQALRDQFGRDNVFLNYR

SEQ ID NO: 12 - protein, 20 aa, Salmonella typhimurium
RLLNDLRGLIGSEQVELEFD

SEQ ID NO: 13 - protein, 20 aa, Staphylococcus aureus
DELGSLPNLPDKAQLSIFDM

SEQ ID NO: 14 - protein, 20 aa, Streptococcus pneumoniae
MGILGNMPEDNQLSLFDELF

SEQ ID NO: 15 - protein, 22 aa, Streptococcus pyogenes
DEMGILGNMPEDNQLSLFDDFF

SEQ ID NO: 16 - protein, 20 aa, Enterococcus faecalis
ENGVLKDLPDENQLSLFDML

SEQ ID NO: 17 - protein, 20 aa, Mycobacterium tuberculosis
PSPALMGDLKELLGPGCLGS

SEQ ID NO: 18 - protein, 371 aa, Ralstonia eutropha
MQLVKTSRDNLLRPLQIVSGIVERRHTLPILANLLIRKSGSNVSFLSTDIEIQITTHAECGVGNDSVATTVAARKLLDILRAMPDGDVALSLNDKRMTVQSGKSRFALQTLAAEEFPTVAEASEFNASVSLPQKTFKHLLAMVHFAMAQQDIRYYLNGMLLVVDGKKVMAVATDGHRLAYCGVELENEATGVGSRQEVIIPRKTILELQRLLEDNDDPVQVQLAANQVKFSFANIELISKLVEGKFPDFQRVIPKGYKNAFAIDRVRLQQALQRTAILTTDKFKGVRCILDTHMLKISSTNADQEEAQEELEIDYSGDALDIGFNVTYLLDVLANLKSEQVQVSLGDSNSSALITVPEDDNFKYVVMPMRI

SEQ ID NO: 19 - protein, 369 aa, Bordetella pertussis
MQLVQTTRDALLKPLSTVAGIVERRHTLPILANILMRKEGNKVAFIATDLEVQITTHADFGVGPENESTTVAARKLLDILKALPDTGEVKLGLASNKLSVQSAKSRFALQTLAASEFPTVAQPEQWDVSLSMPQRMLRHLFNMVHFAMAQQDIRYYLNGMLLVFEPGRVRAVATDGHRLAHCATEADGIAERHEVIVPRKTVLEMQRLLEDSDEPVALDVAPGQIRFRFGDVELVSKLVEGKFPDFTRVIPTNYTRQFSVSREALQGSLQRAAILTTDKFKGVRLQLAQHQMKISSSNAEQEEAQEEIDIDYSFEPLDVGFNVSYLLDVLSNIKAENVQWSVMPDANASALITLPDDEQFKYVVMPMRI

SEQ ID NO: 20 - protein, 367 aa, Neisseria meningitidis
MLILQAERDSLLKPLQAVTGIVERRHTLPILSNVLIEGKGGQTKLLATDLEIQIDTAGPEGGADDFRITTNAKKFQDILRALPAGALVSLDWDDSRLTLKAGKSRFALQTLPAADFPMMNVGEDISATFSLEQERFKTMLSQVQYSMAVQDIRYYLNGLLMQVEGSQLRLVATDGHRLAYAACAIDADLPRAEVILPRKTVLELFKLLNNPDDPIQIELLDKQVRFQCNGTTIVSKVIDGKFPDFNRVIPLDNDKIFVLSRAELLGALERVSILANEKFRGARLFLQSGLLSVVCSNNEQEEAREEIEIAYQGGELEVGFNIGYLMDVLRNIHSDDMQLAFGDANRSTLFTVPNNPNFKYIVMPMRI

SEQ ID NO: 21 - protein, 356 aa, Thiobacillus denitrificans
MSALSAVVGVVERRHTLPILSNLLLEKKAGTLTLLATDLELQVSTRLEAQGGDDFAITIAARKLFDIVRALPDSASVKLDTKDSQVVVSAGKSRFTLQTLPAADFPRVETGAGVGTVVRLPQKTLKRLLQLVQFAMASQDIRYYLNGMLLVLDGQQLRVVATDGHRLSYAETTLDAPTDAQEVIIPRKTVIELSKLLSDSDEPVELRIGSNQVTIMLPGTELVTKVVDGKFPDYQRVIPANQPRHLKANRQTVVQALQRAAILSNEKFRGVRLVMSDNTLGIVCNNNEQEEAADEIEVSYNGDPLDVGFNVTYLLDGLGAVNTDEITLSLGDANSSMLLTSEGEPGFKYVVMPMRI

SEQ ID NO: 22 - protein, 364 aa, Artificial Sequence
  Genbank Accession No. gi/44596445
MNIIINSEEFVSHLNNIVGVVDRKQTMPILGHILMSGMSGEITITATDLEVQISSSFKANITEDFSITLPGRKLFDILRSLGNTDVELSTDNDLVILKTDKSKFSLQQLPANEFPLFDNNESDQSFSIAQSDLSAVFNKTQFAMAQQDVRFYLNGLLLEIKPEKLNVVGTDGHRLAKTNIEIDKKNINEQSCIVPRKAVQELTRSLSDKKDCKISLVDNQASFAFSSVTLTTKLIDGTFPDYNRVIPSETETNILLDTKVLKPALQRVSILANEKFKGVRIDIDNNRITISSENPEQEQAVEDLDIDNTDTKLSIGFNVSYLIDAVNACPGELVALGVNDENTSALITDPSDPNTKYVVMPMRL

SEQ ID NO: 23 - protein, 366 aa, Photorhabdus luminescens
MKFIIEREQLLKPLQQVSSPLSGRPTLPILGNLLLQVTEGSLLLTGTDLEMEMMARVTLSLPHEVGATTVPARKFFDIWRGLPDGAEISVELDGDRLLVRSGRSRFSLSTLPASDFPNLDDWQSEVEFSLPQATLKRLIESTQFSMAHQDVRYYLNGMLFETEGEELRTVATDGHRLAVCSMGIGQKLPSHSVIVPRKGVIELMRLLDGGDTPLQLQIGSNNIRAHVGDFIFTSKLVDGRFPDYRRVLPKNPDKTLEANCDMLKQAFSRAAILSNEKFRGVRLYFSENQLRITANNPEQEEAEEIVDVSYQGAEMEIGFNVSYVLDVLNTLRCEEVRLLLTDAISSVQIEDCANHNAAYVVMPMRL

SEQ ID NO: 24 - protein, 366 aa, Vibrio cholerae
MKFTIERSHLIKPLQQVSGTLGGRASLPILGNLLLKVEENQLSMTATDLEVELISRVTLEGEFEAGSITVPARKFLDICRGLPDSAVITVLLEGDRIQVRSGRSRFSLATLPASDFPNIEDWQSEVQVSLTQAELRGLIEKTQFSMANQDVRYYLNGMLFEIDGTTLRSVATDGHRMAVAQAQLGADFAQKQIIVPRKGVLELVKLLDAPEQPVVLQIGHSNLRAEVNHFVFTSKLVDGRFPDYRRVLPQHTSKTLQTGCEELRQAFSRAAILSNEKFRGVRVNLADNGMRITANNPEQEEAEELLDVSFEGEPIEIGFNVSYILDVLNTLRCDNVRVSMSDANASALVENVDDDSAMYVVMPIRL

SEQ ID NO: 25 - protein, 366 aa, Haemophilus influenzae
MQFSISRENLLKPLQQVCGVLSNRPNIPVLNNVLLQIEDYRLTITGTDLEVELSSQTQLSSSSENGTFTIPAKKFLDICRTLSDDSEITVTFEQDRALVQSGRSRFTLATQPAEEYPNLTDWQSEVDFELPQNTLRRLIEATQFSMANQDARYFLNGMKFETEGNLLRTVATDGHRLAVCTISLEQELQNHSVILPRKGVLELVRLLETNDEPARLQIGTNNLRVHLKNTVFTSKLIDGRFPDYRRVLPRNATKIVEGNWEMLKQAFARASILSNERARSVRLSLKENQLKITASNTEHEEAEEIVDVNYNGEELEVGFNVTYILDVLNALKCNQVRMCLTDAFSSCLIENCEDSSCEYVIMPMRL

SEQ ID NO: 26 - protein, 366 aa, Pasteurella multocida
MQFIVSRENLLKPLQQVCGVLSSRPNIPVLNNVLLQIRGERLVITGTDLEVELSTETALLRTDVQGSFTIPAKKFLDICRSLPEEAEISVSFEEDRAIVKSGRSKFNLSTLPAEEYPNLTDWQSEVDFTLEQSTLRRLIEATQFSMANQDARYFLNGMKFETEGNLLRTVATDGHRLAVCTIALEQDLQTHSVILPRKGVLELVRLLENTDLPARLQIGTNNLRIDLGNIIFTSKLIDGRFPDYRRVLPRNATRILEAEWDVLKQAFVRAAILSNERFRSVRLQLSENQLKITATNPEQEVAEEIIDVSYVGEEMEVGFNVSYILDVLNALKCQRVRMRLTDASSSCLIEDCDDASAEYVIMPMRL

SEQ ID NO: 27 - protein, 367 aa, Actinobacillus pleuropneumoniae
MQFTITREQLLKPLQQVCGVLSSRPTLPVINNILLEIEGDRLSLTGTDLEVELTTVATLEQAVDFAGKFTIPAKKFLDICRSLPEDSSISVQFEEDRALVRAARSKFNLATLPASDYPNLMDWKPEVDFSIEQSTLARLIDATQFSMANQDARYFLNGMKFETEGNLLRTVATDGHRLAVCTMALNQELLTHSVIVPRKAVLELSRLVAVSDETVRLEIGTSNLRVSMNGVVFTSKLIDGRFPDYRRVLPRNADRILEAETEVLKRALMRAAILSNEKFRGVRLALSENLLKITANNPEQEEAEEIIDVAYQSPEMEVGFNVSYLLDVLNTLKCERVRFNLVDASSSCLIEDCDNSTAEYVIMPMRL

SEQ ID NO: 28 - protein, 366 aa, Shewanella oneidensis
MKFSIDRDALLKPLQLVCGAVERRHNLPILANLLVEVSGHSLKLTGTDLEVELVGQAVIHGDIEEGRTTVPAKKLLDIVKSLPEQSELKVEQQDNKWLLRSGRSRFSLATLPAEEYPNVEAFQAEIEFTLKQGVLKSLIDATQFSMANQDVRYYLNGLLIETEGNMLRAIATDGHRLALSHRVIEAQLPEKQVIVPRKGVMEMARLLETDDLDIAISIGDNAIRATTSTTVFTSKLVDGRFPDYRRVLPKGGDKIVIASRNHFKQALTRASILSNEKFRGVRIQLEAGLLKITANNPEQEEAEEIIDVDYNNLPLEIGFNVSYLLDVLNNLKSDDVRITLIDGNSSALLENHLEEDSMYVVMPMRL

SEQ ID NO: 29 - protein, 367 aa, Pseudomonas syringae
MHFTIQREALLKPLQLVAGVVERRQTLPVLSNVLLVVQGQQLSLTGTDLEVELVGRVQLEEPAEPGEITVPARKLMDICKSLPNDALIDIKLDDSKLIVKAGRSRFTLSTLPANDFPTVEEGPGSLTFNLVQSKLRRLIERTSFAMAQQDVRYYLNGMLLEVSAGILRAVATDGHRLAMCSMSADIEHADRHQVIVPRKGILEMARLLTEQDGMVSIVLGQHHIRATTGEFTFTSKLVDGKFPDYERVLPKGGDKLVLGDRQSLREAFSRTAILSNEKYRGIRLQLANGQLKIQANNPEQEEAEEEIGVEYNGEALEIGFNVSYLLDVLSVMTTEQVRLILSDSNSSALVQESDNDDSAYVVMPMRL

SEQ ID NO: 30 - protein, 367 aa, Azotobacter vinelandii
MHFTIQREALLKPLQLVAGVVERRQTLPVLSNVLLVVEKQQLSLTGTDLEVELVGRVPLEENAEPGEITVPARKLMDICKSLPNDTLIDIRLDEQKLLIKAGRSRFSLSTLPASDFPTAEEGLGSLTFSLGQSKLRRLIERTSFAMAQQDVRYYLNGMLLEMNGGVLRAVATDGHRLALCSMQSGIEHADRHQVIVPRKGILELARLLTDQDGEVSIVLGQYHIRATTGEFTFTSKLVDGKFPDYERVLPRGGDKKVLGDRQLLREAFSRTAILSNEKYRGIRLQLASGLLKIQANNPEQEEAEEEVAVDYSGDALEIGFNVSYLLDVLGVMSAEQVCLTLSDSNSSALLQEADNDDSAYVVMPMRL

SEQ ID NO: 31 - protein, 369 aa, Coxiella burnetii
MKLNLIRETLLKPLQLVIGVVERKQTLPILSNVLLSIETDQLSITGTDLEVELIGQTKLDKNSTESSRLTLPGRKLMDICRALPDNAPIELYRDKEKIILRSGRSRFMLSTLPSEDFPAVERRESQLELALPQSTFRHLLYRTHFAMAQQDVRYYLNGLLLETYPNKLRAIATDGHRLAANTLLVQTNTEHRLQIIIPRKGIIELLRLLEEDEALATIRIGNNHIGVSTNDFTFTSKLIEGRFPDCERVIPKGGDKQFVIDRDILKQALSRTAILCNEKFKGVRFELRQGLLRILATNPEQEAAEEEINIDYTGENLDIGFNVGYVLDILNVVNSGNIRLTFSTADSSVLINEVENPSDSAFVVMPMRL

SEQ ID NO: 32 - protein, 382 aa, Acinetobacter sp.
MRLKIAKESLLNVLSHVVGAVERRHTLNILSNVKIQANAQALTITGSDLEVELVASTTLAEGACIEAGETTVPARKLVDICKSLPSAALIDLQITEDQRCILKSGNSRFVLGTLPAEDYPLLTTESSQGTQVQVTQRELKRLFEKTSFAMAVQDVRFYLTGTLLEIDQNQLRAVTTDGHRLALCEVQASSTAMQAVQAIVPRKAVGELQRLLSIEDDQLSLLIGRELLNVTINIANRDKEQHPITVRFTTKLIDGKFPDYRRVIPRGGDKHVQIAHDVFKQSLQRVAILSNEKLRGVFLNFNPDVLQLRANNPEQDEAIEDIAIQYQDASLEMSFNAQYLLDVLSVLDGDDVSMSMTEANQSVLVQDAAHPDQTYVVMPMRV

SEQ ID NO: 33 - protein, 372 aa, Agrobacterium tumefaciens
MRITLERSNLLKSLNHVHRVVERRNTIPILSNVLLRASGANLDMKATDLDLEITEATPAMVEQAGATTVPAHLLYEIVRKLPDGSEVLLATNPDGSSMTVASGRSKFSLQCLPEADFPDLTAGTFSHTFKLKAADLKMLIDRTQFAISTEETRYYLNGIFFHTIESNGELKLRAVATDGHRLARADVDAPSGSEGMPGIIIPRKTVGELQKLMDNPELEVTVEVSDAKIRLAIGSVVLTSKLIDGTFPDYQRVIPTGNDKEMRVDCQTFARAVDRVSTISSERGRAVKLALTDGQLTLTVNNPDSGSATEEVAVGYDNDSMEIGFNAKYLLDITSQLSGEDAIFLLADAGSPTLVRDTAGDDALYVLMPMRV

SEQ ID NO: 34 - protein, 372 aa, Sinorhizobium meliloti
MRITLERSNLLKSLNHVHRVVERRNTIPILSNVLLRSDGASLEMKATDLDLEITEATPAQVEQAGATTVPAHLLYDIVRKLPDGSEVRLATNAEGTAMTVASGRSKFSLQCLPQSDFPDLTAGTFSHSFRLKAPDLKMLIDRTQFAISTEETRYYLNGIFVHTVESNGDLKLRAVATDGHRLARADVEAPSGSEGMPGIIIPRKTVSELQKLLDSPDVVVTVEVSDAKIRLTIGSIVMTSKLIDGTFPDYQRVIPASNDKELRVDCQSFSQAVDRVSTISSERGRAVKLALADGQMTLTVNNPDSGSATEELPVGYESDPLEIGFNAKYLLDITGQLTGGEAVFLLADPGSPTLVRDLAAEDALYVLMPMRV

SEQ ID NO: 35 - protein, 397 aa, Brucella melitensis
MRLIDKGRLPLGCSRPGPSERVRFKMRVTLERSNLLKSLNHVHRVVERRNTIPILSNVLLQAEGASLAMKATDLDLEVNEATAAMVEQAGATTVPAHLLYDIVRKLPDGAEVMLSTNPDGGSMSVISGKSSFRLQCLPQSDFPELTAGAFTHSFRIEAQALKRLIDRTQFAISTEETRYYLNGIFFHAIESDGALKLRAVATDGHRLARAELEAPSGTEGMPGIIIPRKTVAELQKLVDVPDVVVTVELSDAKIRFTVGSVVLTSKLIDGTFPDYQRVIPSGNDKKLTIDRQDFAASVDRVSTISSERGRAVKLSIADGQLTLTVNNPDSGSATDELAADYDGDPLDIGFNSKYLLDITGQLSGTDAVFMLADAGSPTLVRDTGDEDVLYVLMPMRV

SEQ ID NO: 36 - protein, 373 aa, Bartonella henselae
MRITVDRNQLLKSLGRVHRVVERRNTVPILSNVLIDADKNGVQLKATDLDLEVTESFVANIKQEGAITVPAHLLYDIVRKLPDGSEIVLSVDMNQASTMSVVSGCANFQLQCLPKVDFPESLPGQFGYRFSLSTSGLKHLLDCTQFAISTEETRYYLNGIYFHVIDDGVLKLRLVATDGHRLAQVDMEAPSGVEGMPGVIVPRKTVGELQKLLSEEVDEDVCVELSETKIRFSIGSVVFTSKLIDGTFPEYQRVIPLGNDKKLIVNRQDFSSAVDRVSTISSDRGRAVKLTIEQGQLKLIVHNPDSGSAEDQLAVTYTSDPLEIGFNSRYLLDIAGQLSSDEMVFMLAEAGAPALIRNNGVADALYVLMPIRV

SEQ ID NO: 37 - protein, 372 aa, Rhodopseudomonas palustris
MKVTVERAQLLKSLSHVHRVVERRNTIPILGNVLVRAENAQLALKATDLDLEVTETLPAETATAGSTTVPAHMFYDIVRKLPDGSQIVLESDGDRSVLAIRAGRSRFTLQTLPESDFPDLAAGEMSHAFRLPASDVKRLIDRTQFAISTEETRYYLNGIYLHTAGSPKASSLRAVATDGHRLAQLDLTLPSGADGMPGVIVPRKTVGEVQRLIEDTEAEIGIELSTGKIRFTLGNVVLTSKLIDGTFPDYGRVIPQNNDKELLVDKKDFEAAVDRVSTISSERGRAVKLALSAGKLVLSVTNPDSGSATEELEVEYASDPLDIGFNSRYLLDIAAQIEGEVAVLKLADPGSPTLIQDRDSKSALYVLMPMRV

SEQ ID NO: 38 - protein, 375 aa, Novosphingobium aromaticivorans
MKATIERATLLRCLSHVQSVVERRNTIPILSNVLIEAAPDSTVRLMATDLDLQIIETMSAVSVEAPGAITVSAHLLFDIARKLPDGSQVSLETSDNRMIVKAGRSRFQLPTLPRDDFPVIVEGDLPTSFEIPARTLAELIDRTRFAISTEETRYYLNGIFLHVSDDPQPVLKAAATDGHRLARFTITRPDGAEGMPDVIVPRKCVGELRKLLEEVLDSAVLIDLSASKIRFTLGGENGVVLTSKLIDGTFPDYSRVIPTGNDKLLKLDPRSFFEGVDRVATIATEKTRAVKMALENDRVTLSVTSPDNGTAAEELPADYSSTGFEIGFNANYLKDILSQIDGDTVELHLADAGAPTLIRKDDKSAALYVLMPMRV

SEQ ID NO: 39 - protein, 372 aa, Rhodospirillum rubrum
MKLTIERTALLKSLGHVQSVVERRNTIPILSNVRMETGESGFSLNATDMDLEIVETVTATTLAPGATTAPAHTLYDIVRKLPDGSQVELSYDGNDGQLVLKAGRSRFSLSCLPVEDFPALSAGEFSHSFSLPATDLRGLIDRTRFAISTEETRYYLNGIYLHATESAGVPVLRAVATDGHRLARAEIPLPAGAAGMPGVIVPRKTVGELRKLIEETGMDITVGLSEQKIRFDFDETRLTSKLIDGTFPDYERVIPTENDKVLEVDCKLFAQAADRVSAISTEKSRAIKVAVAKGTMTLSASSPDAGSAVEEIEAAYQSTSLEIGFNSRYLLDILGQVEGGSVRLTFADAASPTVIRDVADASAVYVLMPMRV

SEQ ID NO: 40 - protein, 373 aa, Silicibacter sp.
MKISIERATLLKAVAQAQSVVERRNTIPILANVLIEAEGSDVMFRATDLDIEVVDKAPAQVERAGATTVAATTLHEIVRKLPDGALVTLDADAASGRLTVEAGRSNFSLATLPREDFPVMATSEYHSNFTANAAMLRRLFDKSKFAISTEETRYYLNGVYMHVATGESGGKALRCVATDGHRLARIDADLPMGAEDMPGVIVPRKTVGELRKLLDEDDMDIAVSVSETKVRFATPNITLTSKVIDGTFPDYTRVIPVGNTRRLEVDASEFAKAVDRVATVSSERSRAVKLQLDEDRLVLSVNAPDSGAAEEELAVAYSDERLEIGFNAKYLLEIANQVDRENAVFMFNSSGDPTLMREGSDESAVYVVMPMRV

SEQ ID NO: 41 - protein, 372 aa, Caulobacter crescentus
MKLTIERAALLKALGHVQSVVERRNTIPILSNILLSAEGDRLSFSATDLDMEIIDEGFAQIDVPGQITAPAHTLYEIVRKLPDGADVSLSFSGDDPRLVIQAGRSRFNLPVLPAGDFPVMSSDGLSSRIAVDTNELIRLIDKTRFAISTEETRYYLNGLYVHTVNEGGETKLRAVATDGHRLALAEMPAPEGAVGIPGVIVPRKTIAEARRLMESAGETVDLQVSPQKVRFEFGAAALTSKVIDGAFPDYMRVIPRDNAKILTLDNDLFAKAVDRVATISAEKSRSVKLAVEPGRITLTVRNMEAGQAVEEVEVDYDGEPFEIGFNARYLLDVCGQIAGPQAEFRFADPASPTLVVDPVDPGVKYVLMPLRV

SEQ ID NO: 42 - protein, 372 aa, Geobacter sulfurreducens
MEFTIDRDTFSRALQKIQGIVEKRNTMPILSNVLIEALEDRIELTATDLEVGMKSSYPTTVASQGKITVSAKKLYEIVKELPDETISFLTKANDYVEIRCGKAKFTIVGLSSEEFPYFPKVNEESFIRIESGLLADMIEKTSYAICFDETKYNLNGTFVKASEEDGRSILRMVATDGHRLSITQREFNGAVSPEMAKGVIFPKKGIFELKKMCEEESTQLSLGFLDNSAVIVKGNTVVVMRLVDGEFPDYTRVVPVANDRIVTVARDPFFHSLRRMSILSSEKFKGIKMDIQESGMVISSSNPELGEASEELDAVFAGDAISIRFNAKYLIDVLAVLDESSVALHLKDELSPAIVRPADGDGFTAVIMPMRL

SEQ ID NO: 43 - protein, 376 aa, Magnetococcus sp.
MEFHVSREPFLKALQRLQSVVEKRNTMPELGNALLEASAEGLTLTATDLEVSMKSHCPAEVESSGAIAVSARKLFEIVRELPQDSLRLRSEAGERLVLTCGRARFTLVGIRADIFPPFPETTQGQSFTLSGPRLAEMIAKTHFAMSQDETRYTLNGILLHLVSAADAELAGENGLVRIVATDTHRLAMAEMALDIPVEESAELIIPRKGVQEIRKLVEEDDEAVELGLDENFIRVSKPGIVLTSKLVSGRFPNYQRVIPTDNPHLLELEKEPLFGVVKRMMVLSHEKSRGIRMALSSDHIKVSAQNPEQEAAEEEMPCSFAGKDMTVGFNARYLQEIVSVANGDTVRMKLRDEESPVLVEEHSATGYLYVLMPMRV

SEQ ID NO: 44 - protein, 377 aa, Staphylococcus epidermidis
MMEFTIKRDYFINQLNDTLKAISPRTTLPILTGIKIDAKENEVILTGSDSEISIEITIPKQVDGEEIVEITETGSVVLPGRFFVDIIKKLPGKEVKLSTNEQFQTLITSGHSEFNLSGLDPDQYPLLPEVSRDDAIQLSVKVLKNIIAQTNFAVSTSETRPVLTGVNWLIQDNELICTATDSHRLAVRKLQLEDESENKNVIIPGKALSELNKIMSDSDEDIDIFFASNQVLFRVGNINFISRLLEGHYPDTTRLFPENYEIKLGINNGDFYHAIDRASLLAREGGNNVIKLSTGNELVELSSTSPEIGTVKEEVNANDVEGGNLKISFNSKYMMDALKAIDNDEVEVEFFGTMKPFILKPKDDDSVTQLILPIRTY

SEQ ID NO: 45 - protein, 378 aa, Oceanobacillus iheyensis
MRFTIQRDKLINGVSNVMKAISARTVIPILTGMKIEVKNHGVTLTGSDSDISIEYYIPIEEDGIVHVENIEEGTIILQAKYFPDIVRKLPESTVDIVVDDQLNVRITSGKAEFNLNGQSAEEYPQLPKVQTENSFELPIDLLKSMIKQTVFAVSTMETRPILTGVNLKLVDNSLSFTATDSHRLARREIPVSNAPIEISQIVVPGKSLNELNKILGDSEETVEISVTNNQILFRTKHLNFLSRLLDGNYPETSRLIPEQSKTKIQLKTKELLGTIDRASLLAKEERNNVVKFNAPGNSMIEISSNSPEVGNVVEEITADQMEGEDVKISFSSKYMIDALKAIEYDEVQIEFTGAMRPFIIRPVGDDSILQLILPVRTY

SEQ ID NO: 46 - protein, 381 aa, Listeria monocytogenes
MKFVIERDRLVQAVNEVTRAISARTTIPILTGIKIVVNDEGVTLTGSDSDISIEAFIPLIENDEVIVEVESFGGIVLQSKYFGDIVRRLPEENVEIEVTSNYQTNISSGQASFTLNGLDPMEYPKLPEVTDGKTIKIPINVLKNIVRQTVFAVSAIEVRPVLTGVNWIIKENKLSAVATDSHRLALREIPLETDIDEEYNIVIPGKSLSELNKLLDDASESIEMTLANNQILFKLKDLLFYSRLLEGSYPDTSRLIPTDTKSELVINSKAFLQAIDRASLLARENRNNVIKLMTLENGQVEVSSNSPEVGNVSENVFSQSFTGEEIKISFNGKYMMDALRAFEGDDIQISFSGTMRPFVLRPKDAANPNEILQLITPVRTY

SEQ ID NO: 47 - protein, 378 aa, Streptococcus mutans
MIKFSINKVFFLQALNATKRAISSKNAIPILSSLKIEVNSQSITLTGSNGQISIENTISAEEENAGLLVTSSGAILLEANFFINIVSSLPDITLDFEEIEQHQVVLNSGKSEITLKGKDVEQYPRLQEVGTNNPLILETKLLKTIISETAFAASTQESRPILTGVHLVLTNHKEFKAVATDSHRMSQRKLTLDHSSDDFDVVIPSRSLREFAAVFTDDIESVEVFFSNSQILFRSEYISFYTRLLEGNYPDTDRLLGNNFETEVVFNTNALRYAMERAHLISNATQNGTVKLEIINNQVTAHVNSPEVGKVNEELDIESLSGNDLTISFNPTYLIEALKALKSETVTIRFISPIRPFTLTPSDNSENFIQLITPVRTN

SEQ ID NO: 48 - protein, 380 aa, Lactococcus lactis
MIKFSINKTAFQNALKITKQAIGSKVTIPALTKLKIEVEEKGITLIGSNGQISIKNFLPADNKDASMLISGTGSVLLEAAFFENVVSQLPEVTLEFIEKEQKQVLLTSGKSEITLKGLDSEIYPHLQEISEGSSLKMKVKVLKEIFTETVFAVSTQENRPIFTGVHLETLSTGELKAVATDSHRMSQRLLPLEDTDLKFDVILPSKSINSFKNVFTNDEEEIEIFISGSQMLFQNETISYYSRLIEGSYPDTNRLIPNEADYTLDLVFDAAQLRHTMDRARLLTVMTTNGTVKLTVSGDSVVTTANSPEVGSVHEELTALSKEGNDLSISFNPEYLIDALKVIKAPEVRIRFISNVRPFTLQPRNEESGFVQLITPVRTN

SEQ ID NO: 49 - protein, 379 aa, Lactobacillus plantarum
MKFTINRSAFIKELNNVQRAISSKTTIPILTGLKLDVNTDAITLTGSDADISIETTIPASDDNNTLVVEDAGSIVLPARFFSEIVKKLPEDTMTVNVVDGFQTQITSGAASFTINGLDPENYPHLPEIDTTNTITLAGDVLKELIGQTVIAVSNQESRPILTGVHFILANGEFLAVATDSHRLSQRRIKLPEANNANYDVIIPGKSLTELSRMIGDNNPDVQMRLSENQVLFVLGNTSFYSRLLEGNYPDTSRLIPKESNTTVEISAPALSAAIERASLLSHESRNNVVRFSVNPTDKTITIFGNSPDVGEVTEQLQPTDLSGDELEISFNPDYMKEALRSFGQAMIKISFTMALRPFTLVPTEEGENFIQLITPVRTF

SEQ ID NO: 50 - protein, 376 aa, Lactobacillus johnsonii
MQFTINRNLFLENLNNAMRAISSRATIPILSGIKLTLTDEMLTLTGSDTDISIEIQIPVNDDLIVQSTGSIVLPARFFSEIVKKLPGKDFSFEVKESFQTKIVSENTEFMINGLDANNYPHLPEISTDASFKISGKTFREIINETVFAVATQESRPTLTGVNFIFNNSSIKAVATDSHRLSQRQISLENGPQTSTDLIIPGKSLVELSRIIGESDPEITVNPGENQVLFEVGNIAFYSRLLDGQYPDTDRLIPTESTTSVEFELPVLARSLERASLLTHESRNNVVKMTLDVQNQLVKLQGDSPEIGNVEEEIGFKNLEGDGLTISFNPDYLREALRASITDSIIMNFTQPLRPFTVIPAKQDVNFTQLITPVRTF